(19) United States
(12) Patent Application Publication
(10) Pub. No.: US 2026/0306776 A1
(43) Pub. Date: Oct. 8, 2026

(54) ORONASAL PATIENT INTERFACE ADAPTED FOR USE IN POSITIVE AIRWAY PRESSURE DELIVERY

(71) Applicant: Leslie HOFFMAN, Tarzana, CA (US)

(72) Inventor: Leslie HOFFMAN, Tarzana, CA (US)

(21) Appl. No.: 19/480,770

(22) PCT Filed: May 3, 2024
(86) PCT No.: PCT/US2024/027676

§ 371 Date: Oct. 31, 2025

Related U.S. Application Data
(60) Provisional application No. 63/499,818, filed on May 3, 2023.
(60) Provisional application No. 63/514,775, filed on Jul. 20, 2023.
(60) Provisional application No. 63/619,689, filed on Jan. 10, 2024.
(60) Provisional application No. 63/561,126, filed on Mar. 4, 2024.

(51) Int. Cl. A61M 16/06; A61M 16/10

(52) U.S. Cl. CPC A61M 16/0666; A61M 16/0605; A61M 16/0683; A61M 16/1045; A61M 2202/0007; A61M 2202/02; A61M 2205/0216; A61M 2205/3303; A61M 2205/3306; A61M 2205/3344; A61M 2209/088; A61M 2210/0618; A61M 2210/0625; A61M 2210/1025; A61M 2230/20

(57) ABSTRACT

An oronasal interface system for administration of positive airway pressure to the airway that comprises a nasal interface portion and an oral interface portion, wherein the oral interface portion encloses the chin of the individual.

The present invention claims the benefit of U.S. Provisional Application No. 63/499,818 filed May 3, 2023; U.S. Provisional Application No. 63/514,775 filed Jul. 20, 2023; U.S. Provisional Application No. 63/619,689 filed Jan. 10, 2024; and U.S. Provisional Application No. 63/561,126 filed Mar. 4, 2024, each of which is hereby incorporated in its entirety and from each of which priority is claimed.

### FIELD OF THE INVENTION

The present invention relates to devices and methods for the treatment of airway disorders using positive airway pressure.

### BACKGROUND OF THE INVENTION

The following discussion of the background of the invention is merely provided to aid the reader in understanding the invention and is not admitted to describe or constitute prior art to the present invention.

Positive airway pressure (PAP) therapy is a generic term applied to treatments using a stream of air at greater than atmospheric pressure to support airway and pulmonary patency. Typical PAP therapy involves a portable machine that directs pressurized room air into the airway through a tube connected to a face mask. This positive airflow helps keep the airway open. The most familiar application of PAP therapy is preventing the collapse that occurs during sleep apnea, thus allowing normal breathing.

There are various types of PAP therapy that may be employed. Continuous positive airway pressure (CPAP) is a type of PAP therapy in which the air flow is introduced into the airways to maintain a continuous pressure to constantly stent the airways open, in people who are breathing spontaneously. In bilevel positive airway pressure (BiPAP), the air pressure cycles between two levels of pressure based on whether the patient is inhaling or exhaling, but positive pressure is delivered continuously at some level. These pressures are known as inspiratory positive airway pressure (IPAP) and expiratory positive airway pressure (EPAP).

The pressures involved are typically recited in terms of centimeters of water pressure (cm H₂O) because lung pressure is often expressed in those units. 1 cm H₂O is the amount of pressure required to raise a column of water 1 cm in height. This is equal to 98.07 Pascals. The pressures utilized in PAP therapy are generally between 2 and 30 cm H₂O. Various interfaces are available for the delivery of PAP and most can be divided into 3 categories: nasal masks which cover the nose and leave the mouth free; nasal pillows which insert directly into the nostrils; and oronasal (i.e., full face) masks, which cover the nose and the mouth. These interfaces are collectively referred to herein as “face masks.”

Patient interfaces, such as a mask assembly, for use with blowers and flow generators in the treatment of sleep disordered breathing (SDB) typically include a soft patient-contacting portion, such as a cushion, and a rigid shell or frame. In use, the patient interface is held in a sealing position by headgear so as to enable a supply of air at positive pressure to be delivered to the patient's airways. When the cushion and frame are manufactured from different materials, they need to be held together in some way. It is also generally desirable that the patient interface be cleanable, for example, allowing a person to wash a mask between uses. While some semi-permanent assembly methods are available, they generally leave small gaps and crevices that can accumulate dirt and be difficult to clean. Hence, it is generally desirable that the frame and cushion include a mechanism that enables both assembly and disassembly. It is also desirable that there be a seal between the frame and cushion to reduce or eliminate leaks from the assembly in use. Since many patients lack dexterity, a good design is simple and easy to use for patients. A number of cushion-to-frame assembly mechanisms are known. For example, see U.S. Pat. No. 6,412,487 to Gunaratnam, et al. and U.S. Pat. No. 6,823,869 to Raje et al. Also see ResMed's MIRAGE®, ULTRA MIRAGE®, ACTIVAR, and VISTA® masks.

Although mouth breathing is common among OSA patients, a nasal mask is reportedly the best option for most patients. Genta et al., Ann Am Thorac Soc. 2020 October; 17(10): 1177-1185 (doi: 10.1513/AnnalsATS.202007-864ST). Mouth leak can occur if respiratory therapy is delivered to the patient's nasal cavity with a nasal patient interface but the mouth of the patient is open or opens during delivery of the therapy. The patient's open mouth may provide a pathway for the pressurized gases to bypass the patient's lungs. The presence of a mouth leak may effectively render the respiratory support system unsealed due to gases being able to leave the patient's mouth to atmosphere. This can unintentionally reduce the pressure of gases in the respiratory support system below a desired pressure level. The sub-optimal pressure level may compromise the efficacy of the respiratory support. Headgear, such as a chin restraint, may be fitted to a patient and can be used to urge the mouth of the patient closed to reduce mouth leak and ensure that gases are delivered at to the patient at the desired pressure level. But chin restraints may be uncomfortable for the patient, inconvenient to don, and/or inconvenient to adjust, for example.

Oronasal masks have been described, such as the Philips Respironics Dream Wear full face mask, in an attempt to deal with such issues common to nasal masks. These oronasal masks, however, suffer from issues with comfort, a requirement for higher therapeutic pressures, and poorer acceptance by patients. In studies comparing nasal versus oronasal CPAP, nasal interfaces resulted in better patient compliance, reduced sleepiness, and lower residual apnea-hypopnea index. Mortimore et al., Thorax. 1998; 53:290-292; Ebben et al., Sleep Med. 2014; 15:619-624.

### BRIEF DESCRIPTION OF THE INVENTION

It is an object of the invention to provide a method and apparatus for a patient interface for use in delivery of gases under positive pressure to an individual, for example in the treatment of sleep disordered breathing (SDB) such as obstructive sleep apnea (OSA). It is another object of the invention to provide a body-worn PAP system providing proper mask fitment during body movement, reduced leakage, and elimination of a need for a connecting hose to the blower unit. It is a second object of the invention to provide a PAP blower adapted for use in a PAP system with a compact noise reduction system that does not need acoustic foams in the airflow path.

In a first aspect, the invention relates to an oronasal patient interface adapted for the delivery of positive air pressure to an individual, comprising:
a nasal interface portion comprising a first resilient elastomeric hollow chamber body defining a first space-filled interior chamber, the first hollow chamber body comprising
   a concave, bowl-shaped, skin contact surface configured to receive the nose of the individual, wherein the skin contact surface comprises a superior margin configured to contact the individual's skin at the supratip lobule and terminate contact of the unitary chamber with the individual's skin at or inferior to the supratip break, and an inferior margin configured to contact the individual's skin inferior to the coumella and superior to the upper vermillion border,
   a surface recess in the skin contact surface positioned inferior to the superior margin and configured to receive the individual's tip lobule,
   a pair of nasal interfaces, each nasal interface comprising an opening at an upper surface thereof in fluid communication with the space-filled interior chamber, and
   first and second elastomeric side members, each side member configured to extend laterally from the resilient elastomeric hollow chamber body and comprising an interior surface recess in the second surface region configured to receive the individual's alar rim, wherein each side member is configured to contact the individual's skin and extend over a nasal ala towards a position overlying the maxilla and terminate contact of the first hollow chamber body with the individual's skin proximal to the zygomaticomaxillary suture;
an oral interface portion configured to define a plenum space over the mouth of the individual, the oral interface comprising a perimeter surface providing a seal-forming structure configured to form a seal with a region of the patient's face surrounding an entrance to the patient's airways and extend around and under the mental protuberance of the individual to envelop said mental protuberance; and
a gas ingress port configured to operably connect to a source of pressurized gas,
   wherein the plenum space and the hollow chamber body are each configured in fluid communication with the gas ingress port and with each other such that pressurized gas entering the unitary apparatus through the gas ingress port is delivered to the individual's airway via the plenum space and the nasal interfaces in the hollow chamber body.

The term “plenum space” as used herein refers to a chamber configured to contain a pressurized gas for delivery to the airway of an individual via an oral route. As will be understood by one of skill in the art, an oral mask forms a seal on the face of the wearer, thereby defining an enclosed air-filled volume within the mask—the “plenum space”—overlying the patient's mouth. As described hereinafter, in the oronasal masks of the present invention, this plenum space is in fluid communication with the hollow chamber body of the nasal interface portion such that the pressurized gas is administered by both the oral and nasal route.

In the oronasal masks of the present invention, the oral interface portion extends around and under the mental protuberance of the mandible in an intimate fashion such that the plenum space formed by the oral interface portion ends below the mental protuberance. This allows the oral interface portion to include an anchor under the mandible which also acts as a gentle chin restraint to discourage mouth leaks.

In certain embodiments each nasal interface is formed as a hollow, approximately conical frustra extending from the skin contact surface and configured to insert into a nostril, wherein each nasal interface. In certain other embodiments each nasal interface is an opening in the surface of the hollow chamber body configured to abut the wearer's nose at the location of each nostril.

In certain embodiments, a headgear is configured to firmly hold the oral interface portion around and under the mental protuberance of the mandible so that the oral interface portion is pulled upward against the underside of the mandible. By extending around and under the mental protuberance of the mandible, this upward force from the headgear secures the anchor of the oral interface portion under the mandible, making the oronasal patient interface less prone to dislodge from its seated position on the face of the individual. At the same time, this upward force acts to secure the nasal interface portion against the underside of the nose, reducing the possibility of air leakage and further limiting the ability of the oronasal patient interface to be dislodged from the face of the wearer.

In preferred embodiments, the nasal interface and the oral interface portions of the oronasal patient interface of the present invention are formed as a combined single, unitary piece, typically of a suitable elastomeric material as described hereinafter. The term “unitary” as used herein with reference to an element of the oronasal interface system refers to the element as being formed of a single discrete piece. This contrasts with “integral” which, as used herein, refers to the element as being “composed of integrated parts.” By way of example, the nasal interface and the oral interface portions may be constructed as a unitary piece, but that unitary piece may subsequently mate to a separately constructed substantially rigid housing (also referred to herein as a “base” or “frame”) to form an integral unit.

In certain embodiments, the fluid connection between the nasal interface portion and the oral interface portion comprises one or more openings below the lower boundary of nasal interface portion. Examples of such a fluid connection are shown in FIGS. 3 and 9B. In the case where the fluid connection comprises at least two spaced apart openings, the portion of the unitary elastomeric device comprises a region of regional stiffness between the two openings to maintain the stiffness of the nasal interface portion

In various embodiments, the oronasal interface system further comprises a unitary substantially rigid housing comprising:
a first surface configured to mate to the gas ingress port and a second surface configured to operably connect to a gas source, thereby providing a fluidly connected pathway between the gas source and the gas ingress port. In certain embodiments, the unitary substantially rigid housing further comprises shielding portions extending across a portion of the external surface of the chamber body distal to the skin contact surface.

In certain embodiments, first and second solid elastomeric side members of the nasal interface portion each further comprise an interior surface recess in the second surface region configured to receive the individual's alar rim.

In certain embodiments, the fourth surface region of the nasal interface portion is configured to provide a region of increased flexibility in the portion of the side member overlying the maxilla by introducing a recessed area in the fourth surface region that results in thinning of the material forming the solid elastomeric side member. In addition to the thinned area being more flexible than the relatively thicker areas of the solid elastomeric side member, the transition between the thicker areas and the thinned area provides a hinging action at the transition point. This can enhance comfort for the wearer while still maintaining the position and stability of the oronasal interface system on the face.

In certain embodiments, the oronasal interface systems comprise (i) one or more exhaust apertures in the nasal interface portion and/or the oral interface portion that fluidly connect to ambient external atmosphere; (ii) one or more exhaust apertures through the substantially rigid housing that fluidly connect the space-filled interior chamber to ambient external atmosphere; or (iii) both (i) or (ii). The term “exhaust aperture” as used herein refer to discrete openings engineered into the oronasal interface systems to permit gas from the interface system to escape during exhaling. This is not meant to indicate that such apertures only operate to remove exhaled air. During inspiration the oxygen-enriched air that has accumulated in the mask, along with some room air entrained through the exhaust apertures, is inhaled. During expiration, exhaled gas is deposited in the mask, with the rest exiting through the vent holes. The continuous flow of air from the air source then partially washes out the mask before the next inspiration.

As described hereinafter, one or more of the exhaust apertures comprise a flow path length of from about 3 mm to about 7 mm. Thus, these apertures are in the form of elongate hollow tubes. The term “flow path” as used in connection with the tubular exhaust apertures refers to the distance from one open end of the tube to the other open end. Such apertures may be in the chamber body, but are preferably in the substantially rigid housing. There may be any number of such tubular exhaust apertures, with preferred numbers being between 4 and 60, more preferably between 10 and 30, still more preferably between 15 and 25, and in certain embodiments 15, 16, 17, 18, 19, or 20. In various embodiments, these tubular exhaust apertures comprise an open bore diameter of between about 0.7 mm to about 2.5 mm, and are preferably not tapered (meaning the open bore diameter of a tubular exhaust aperture does not vary along its length). In certain embodiments, the flow path through a tubular exhaust aperture is substantially linear (meaning the tubular exhaust aperture does not comprise a substantial change in angle along its length). Most preferably, the open bore diameter of a tubular exhaust aperture is about 0.8 mm, about 1.0 mm, about 1.2 mm, about 1.4 mm, about 1.6 mm, about 1.8 mm, or about 2 mm.

In certain embodiments, the one or more exhaust apertures provide a total aperture area of between about 15 mm² to about 30 mm². By “total aperture area” is meant the sum of the surface area of all exhaust apertures in the oronasal interface system, measured at the exterior opening of each exhaust aperture. By way of example, if the number of exhaust apertures in the unitary substantially rigid housing is 17 and the open bore diameter of each of the exhaust apertures in the unitary substantially rigid housing is about 1.2 mm, the total aperture area would be 17(π)(0.6²)=19mm².

The term “unitary” as used herein with reference to an element of the nasal interface system refers to the element as being formed of a single discrete piece. This contrasts with “integral” which, as used herein, refers to the element as being “composed of integrated parts.” By way of example, the unitary nasal interface system may be integral with regard to a substantially rigid housing, wherein the unitary chamber is configured to mate to the substantially rigid housing to form a single integral unit.

The term “resilient” as used herein with regard to the oronasal interface refers to the material of which the unitary chamber is composed being able to substantially retain its shape at ambient air pressure and in the absence of a pressure differential within the chamber versus outside the chamber, but being able to be easily deformable by an applied pressure such as a pinch between two fingers that applies a pressure of 4 N to the surface of the unitary chamber. In certain embodiments, certain portions of the oronasal interface may be more or less resilient than other portions. For example, those parts of the oral interface portion lying under the mandible may be made of a thicker or more dense material than those parts forming the peripheral seal around the mouth or forming the nasal interface portion. In certain embodiments, the portion of the chamber body forming the space-filled interior chamber may be made both softer and thinner than the gas ingress port, so that the space-filled interior chamber provides comfort to the wearer and the gas ingress port provides a more positive mating surface to the unitary substantially rigid housing. In certain embodiments, the portion of the chamber body forming the space-filled interior chamber of the nasal interface portion has a Shore A durometer of between 10 and 80. By way of example only, the portion of the chamber body forming the space-filled interior chamber may have a Shore A durometer of between 30 and 60 and a wall thickness of between 0.5 mm and 2.0 mm.

The term “elastomeric” as used herein refers to an amorphous polymeric material that is maintained above their glass transition temperature, so that considerable molecular reconformation is feasible without breaking of covalent bonds. At ambient temperatures, such elastomeric materials are thus relatively compliant (E≈3 MPa) and deformable. Examples of elastomeric materials include, but are not limited to, silicone elastomers, polyurethane elastomers, and polyurethane siloxane copolymer elastomers.

The term “substantially rigid” as used herein refers to a material that having the character of being able to self-maintain a stiff, three-dimensional shape in the absence of any external force being applied, but which may bend to some degree without breaking when a sufficient external force is applied. That is, a “substantially rigid” material may be rigid enough to provide its own structural support without being brittle. For example, thick glass may be a rigid material, whereas some types of plastic may be substantially rigid materials. This contrasts with the term “flexible” which, as used herein, refers to materials that are pliable and capable of easily bending without breaking when lesser amounts of external forces are applied, and rely on an external support to maintain a three-dimensional structure.

In certain embodiments, the oronasal interface system may further comprise a hygroscopic condenser humidifier element, which may preferably be positioned within the unitary substantially rigid housing, configured to maintain a level of the humidity of the inhaled gas as it traverses the fluidly connected pathway between the gas source and the user. By way of example, the hygroscopic condenser humidifier element may preferably be positioned within the unitary substantially rigid housing upstream of the gas ingress port. Such a hygroscopic condenser humidifier can act to retain moisture in gases exhaled by the wearer and return this moisture to the inhaled gas stream.

Because of its soft, thin construction, introduction of positive air pressure in the range of 4-20 cm H₂O into the hollow chamber body can cause the thin parts of the hollow chamber body (e.g. <2 mm thick, preferably <1.5) to inflate slightly, thereby increasing the space-filled chamber volume of the hollow chamber body of the nasal interface portion or to provide an air-cushioning effect in use. This tendency to expand can serve to improve sealing of the oronasal interface system to the wearer's airway by causing each skin-contacting area to adapt somewhat to the wearer's anatomy and firmly support the approximately conical fustra shape within the nostril. The pliability of the inflated chamber spreads the load evenly on the user's skin contact area minimizing pressure points and enhancing comfort. The choice of material for the hollow chamber body can act to limit this expansion so that the unitary chamber doesn't shift on the face during normal movement of the wearer.

In certain embodiments, the opening at the upper surface of each nasal interface through which pressurized air is administered to the wearer is oval, stadium, pyriform, spherical, or elliptical in shape.

In certain embodiments, the oronasal interface system is configured such that the skin contact surface of the unitary chamber is configured to terminate proximal to or upon the infraorbital portion of the face of the individual between the superior and inferior margins. In this manner, the side edges of the chamber body rest upon, or within 1-5 mm of, the cheeks of the wearer.

In a related aspect, the present invention relates to methods of treating sleep apnea, snoring, or another breathing disorder in an individual, comprising: positioning the oronasal interface system over the mouth and nose of the individual such that the nasal interfaces, each formed as a hollow, approximately conical, frustra extending from the skin contact surface, insert into the nostrils of an individual and the lower portion of the oral interface portion envelopes the mental protuberance; and energizing a flow generator operably connected to the oronasal interface system to administer airflow produced by the flow generator into the individual's mouth nostrils via the oronasal interface system.

In another aspect, the invention relates to nasal interface systems for delivery of positive air pressure to an individual, comprising:

a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
wherein the first surface region comprises
   a superior margin configured to contact the individual's skin at the supratip lobule and terminate contact of the unitary chamber with the individual's skin at or inferior to the supratip break,
   an inferior margin configured to contact the individual's skin inferior to the coumella and terminate contact of the unitary chamber with the individual's skin at or superior to the upper vermillion border,
   a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
   a surface recess in the first surface region positioned inferior to the upper margin and configured to receive the individual's tip lobule; and
first and second hollow elastomeric side members, each side member configured to extend laterally from the resilient elastomeric hollow chamber body such that the hollow interior of the elastomeric side members is fluidly continuous with the hollow interior of the hollow chamber body, wherein each side member comprises
   a third surface region configured to contact the individual's skin and extend over a nasal ala towards a position overlying the maxilla and terminate contact of the unitary chamber with the individual's skin proximal to the zygomaticomaxillary suture, and
   an interior surface recess in the second surface region configured to receive the individual's alar rim; and
one or more exhaust apertures through the chamber body that fluidly connect the space-filled interior chamber to ambient external atmosphere.

In a related aspect, the invention relates to nasal interface systems for delivery of positive air pressure to an individual, comprising:
a unitary chamber comprising:
a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
   wherein the first surface region comprises
   a superior margin configured to contact the individual's skin at the supratip lobule and terminate contact of the unitary chamber with the individual's skin at or inferior to the supratip break,
   an inferior margin configured to contact the individual's skin inferior to the coumella and terminate contact of the unitary chamber with the individual's skin at or superior to the upper vermillion border,
   a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
   a surface recess in the first surface region positioned inferior to the upper margin and configured to receive the individual's tip lobule; and
first and second solid elastomeric side members, each side member configured to extend laterally from the resilient elastomeric hollow chamber body, wherein each side member comprises
   a third surface region configured to contact the individual's skin and extend over a nasal ala towards a position overlying the maxilla and terminate contact of the unitary chamber with the individual's skin proximal to the zygomaticomaxillary suture,
   an interior surface recess in the second surface region configured to receive the individual's alar rim, and
   a fourth surface region configured to face distally from the individual's skin and to provide a region of increased flexibility in the portion of the side member overlying the maxilla.

In certain embodiments, first and second solid elastomeric side members each further comprise an interior surface recess in the second surface region configured to receive the individual's alar rim.

In certain embodiments, the nasal interface system further comprises a unitary substantially rigid housing comprising:
a first surface configured to mate to the gas ingress port and a second surface configured to operably connect to a gas source, thereby providing a fluidly connected pathway between the gas source and the gas ingress port.

In certain embodiments, the unitary substantially rigid housing further comprises shielding portions extending across a portion of the external surface of the chamber body distal to the skin contact surface.

In certain embodiments, the fourth surface region is configured to provide a region of increased flexibility in the portion of the side member overlying the maxilla by introducing a recessed area in the fourth surface region that results in thinning of the material forming the solid elastomeric side member. In addition to the thinned area being more flexible than the relatively thicker areas of the solid elastomeric side member, the transition between the thicker areas and the thinned area provides a hinging action at the transition point. This can enhance comfort for the wearer while still maintaining the position and stability of the nasal interface system on the face.

In certain embodiments, the nasal interface systems comprise (i) one or more exhaust apertures through the chamber body that fluidly connect the space-filled interior chamber to ambient external atmosphere; (ii) one or more exhaust apertures through the substantially rigid housing that fluidly connect the space-filled interior chamber to ambient external atmosphere; or (iii) both (i) or (ii). The term “exhaust aperture” as used herein refer to discrete openings engineered into the nasal interface systems to permit gas from the interface system to escape during exhaling. This is not meant to indicate that such apertures only operate to remove exhaled air. During inspiration the oxygen-enriched air that has accumulated in the mask, along with some room air entrained through the exhaust apertures, is inhaled. During expiration, exhaled gas is deposited in the mask, with the rest exiting through the vent holes. The continuous flow of air from the air source then partially washes out the mask before the next inspiration.

As described hereinafter, one or more of the exhaust apertures comprise a flow path length of from about 3 mm to about 7 mm. Thus, these apertures are in the form of elongate hollow tubes. The term “flow path” as used in connection with the tubular exhaust apertures refers to the distance from one open end of the tube to the other open end. Such apertures may be in the chamber body, but are preferably in the substantially rigid housing. There may be any number of such tubular exhaust apertures, with preferred numbers being between 4 and 60, more preferably between 10 and 30, still more preferably between 15 and 25, and in certain embodiments 15, 16, 17, 18, 19, or 20. In various embodiments, these tubular exhaust apertures comprise an open bore diameter of between about 0.7 mm to about 2.5 mm, and are preferably not tapered (meaning the open bore diameter of a tubular exhaust aperture does not vary along its length). In certain embodiments, the flow path through a tubular exhaust aperture is substantially linear (meaning the tubular exhaust aperture does not comprise a substantial change in angle along its length). Most preferably, the open bore diameter of a tubular exhaust aperture is about 0.8 mm, about 1.0 mm, about 1.2 mm, about 1.4 mm, about 1.6 mm, about 1.8 mm, or about 2 mm.

In certain embodiments, the one or more exhaust apertures provide a total aperture area of between about 15 mm² to about 30 mm². By “total aperture area” is meant the sum of the surface area of all exhaust apertures in the nasal interface system, measured at the exterior opening of each exhaust aperture. By way of example, if the number of exhaust apertures in the unitary substantially rigid housing is 17 and the open bore diameter of each of the exhaust apertures in the unitary substantially rigid housing is about 1.2 mm, the total aperture area would be 17(π)(0.62)=19 mm².

In certain embodiments, the nasal interface system may further comprise a hygroscopic condenser humidifier element positioned within the unitary substantially rigid housing or within the unitary chamber and configured to maintain a level of the humidity of the inhaled gas as it traverses the fluidly connected pathway between the gas source and the user. By way of example, the hygroscopic condenser humidifier element may preferably be positioned within the unitary substantially rigid housing upstream of the gas ingress port. Such a hygroscopic condenser humidifier can act to retain moisture in gases exhaled by the wearer and return this moisture to the inhaled gas stream.

In certain embodiments, the portion of the chamber body forming the space-filled interior chamber may be made both softer and thinner than the posterior surface of the chamber body that comprises the gas ingress port, so that the space-filled interior chamber provides comfort to the wearer and the posterior surface provides a more positive mating surface to the unitary substantially rigid housing. In certain embodiments, the portion of the chamber body forming the space-filled interior chamber has a Shore A durometer of between 10 and 80. By way of example only, the portion of the chamber body forming the space-filled interior chamber may have a Shore A durometer of between 30 and 60 and a wall thickness of between 0.5 mm and 2.0 mm in the sealing regions.

Because of this soft, thin construction in the sealing regions of the interface, introduction of positive air pressure in the range of 4-20 cm H₂O into the hollow chamber body can cause the hollow chamber body to inflate slightly to enhance sealing in the thinned regions or to provide an air-cushioning effect in use. This tendency to expand can serve to improve sealing of the nasal interface system to the wearer's airway by causing each nasal interface to adapt to the wearer's anatomy and firmly support the conical frustral shape within the nostril. Thus, in certain embodiments, introduction of positive air pressure in the range of 4-20 cm H₂O into the hollow chamber body can increase the seal of each nasal interface within the nostril. The pliability of the inflated chamber spreads the load evenly on the user's skin contact area minimizing pressure points and enhancing comfort. The choice of material for the hollow chamber body can act to limit this expansion so that the unitary chamber doesn't shift on the face during normal movement of the wearer and this resistance to movement is enhanced by regionally thicker, stiffer regions of the interface.

In certain embodiments, the opening at the upper surface of each nasal interface through which pressurized air is administered to the wearer is oval, stadium, pyriform, spherical, or elliptical in shape.

In certain embodiments, the nasal interface system is configured such that the skin contact surface of the unitary chamber is configured to terminate proximal to or upon the infraorbital portion of the face of the individual between the superior and inferior margins. In this manner, the side edges of the chamber body rest upon, or within 1-5 mm of, the cheeks of the wearer.

In a related aspect, the present invention relates to methods of treating sleep apnea, snoring, or another breathing disorder in an individual, comprising:
positioning the nasal interface system over the nose of the individual such that the nasal interfaces, each formed as a hollow, approximately conical, frustra extending from the skin contact surface, insert into the nostrils of an individual; and
energizing a flow generator operably connected to the nasal interface system to administer airflow produced by the flow generator into the individual's nostrils via the nasal interface system.

The use of elongate tubular exhaust apertures described herein is applicable generally to patient interfaces for administering gas to a user. Thus, in another aspect, the present invention provides patient interfaces comprising:
a mask configured to cover the nose and/or mouth of a user in use, the mask comprising a resilient user contact surface, a gas ingress port for receiving the gas from a gas source, and one or more administration apertures in the resilient user contact surface for gas administration to the user;
a substantially rigid housing comprising a first surface in fluid communication with the gas ingress port and a second surface in fluid communication with the gas source, thereby providing a fluidly connected pathway at a location between the gas source and the gas ingress port; and
one or more exhaust apertures within the substantially rigid housing that fluidly connect the fluidly connected pathway to ambient external atmosphere, wherein one or more of the exhaust apertures in the substantially rigid housing comprise a flow path length of from about 3 mm to about 7 mm.

As described above, there may be any number of such tubular exhaust apertures, with preferred numbers being between 4 and 60, more preferably between 10 and 30, still more preferably between 15 and 25, and in certain embodiments 15, 16, 17, 18, 19, or 20. In various embodiments, these tubular exhaust apertures comprise an open bore diameter of between about 0.7 mm to about 2.5 mm, and are preferably not tapered (meaning the open bore diameter of a tubular exhaust aperture does not vary along its length). In certain embodiments, the flow path through a tubular exhaust aperture is substantially linear (meaning the tubular exhaust aperture does not comprise a substantial change in angle along its length). Most preferably, the open bore diameter of a tubular exhaust aperture is about 0.8 mm, about 1.0 mm, about 1.2 mm, about 1.4 mm, about 1.6 mm, about 1.8 mm, or about 2 mm. In certain embodiments, the one or more exhaust apertures provide a total aperture area of between about 15 mm² to about 30 mm².

The term “about”, “approximately”, or “approximate”, when used in connection with a numerical value, means that a collection or range of values is included. For example, “about X” includes a range of values that are ±20%, ±10%, +5%, +2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE FIGURES

FIG. 1 depicts facial landmarks referred to in the present specification.

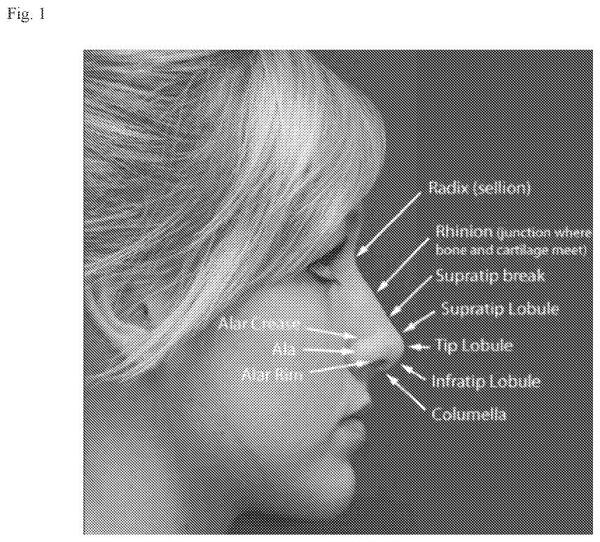

FIG. 2 depicts facial landmarks referred to in the present specification.

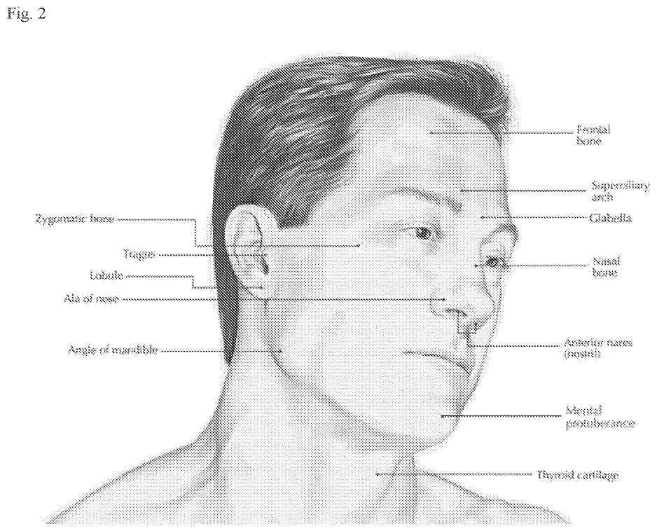

FIG. 3 depicts a 3-D rendering of an elastomeric oronasal interface of the present invention.

FIG. 4 depicts an oronasal interface system of the present invention positioned on an individual.

FIG. 5 depicts an exploded and a side elevation view of an elastomeric oronasal interface of the present invention.

FIG. 6 depicts a unitary substantially rigid housing (or “frame”) of the present invention as an elevation view and a sectional view.

FIG. 7 depicts an oronasal interface system of the present invention as two elevation views and a sectional view.

FIG. 8 depicts an oronasal interface system of the present invention assembled with a frame as an elevation view and a sectional view.

FIG. 9A depicts a 3-D rendering of an alternative oronasal interface system of the present invention.

FIG. 9B depicts a 3-D rendering of an alternative oronasal interface system of the present invention.

FIG. 10 depicts an elevation view of an oronasal interface system of the present invention showing the skin contacting aspect of the interface.

FIG. 11 depicts an elevation view of an oronasal interface system of the present invention showing the upper (relative to the face of the wearer) aspect of the interface and a sectional view.

FIG. 12 depicts a cross sectional view of an oronasal interface system of the present invention and two details of the oronasal interface system.

FIG. 13 an exploded view of of an oronasal interface system of the present invention and its associated frame and headgear.

FIG. 14 depicts a 3-D rendering and various elevation views of a frame adapted for use with an oronasal interface system of the present invention.

FIG. 15 depicts sectional and detail views of a frame adapted for use with an oronasal interface system of the present invention.

FIG. 16 depicts side elevation and detail views of a frame adapted for use with an oronasal interface system of the present invention.

FIG. 17 depicts two elevation and two detail views of an alternative frame adapted for use with an oronasal interface system of the present invention.

FIG. 18 depicts an oronasal interface system of the present invention assembled with a PAP blower as described in PCT/US2022/043556 assembled as an integral unit.

FIG. 19 depicts a nasal interface system of the present invention in assembled and exploded views.

FIG. 20 depicts alternative views of a nasal interface system of the present invention.

FIG. 21 depicts views of a resilient elastomeric chamber body component of a nasal interface system of the present invention with exemplary features shown as angles (in degrees), diameters φ (in millimeters), and linear dimensions (in millimeters).

FIG. 22 depicts views of a resilient elastomeric chamber body component of a nasal interface system of the present invention with exemplary wall thicknesses (in millimeters).

FIG. 23 depicts a nasal interface system of the present invention as a component of a positive airway pressure device as described in PCT/US2022/043556.

FIG. 24 depicts a nasal interface system of the present invention as a component of a positive airway pressure device configured to connect to a PAP blower by a flexible supply conduit in exploded form.

FIG. 25 depicts a nasal interface system of the present invention as a component of a positive airway pressure device configured to connect to a PAP blower by a flexible supply conduit in assembled form.

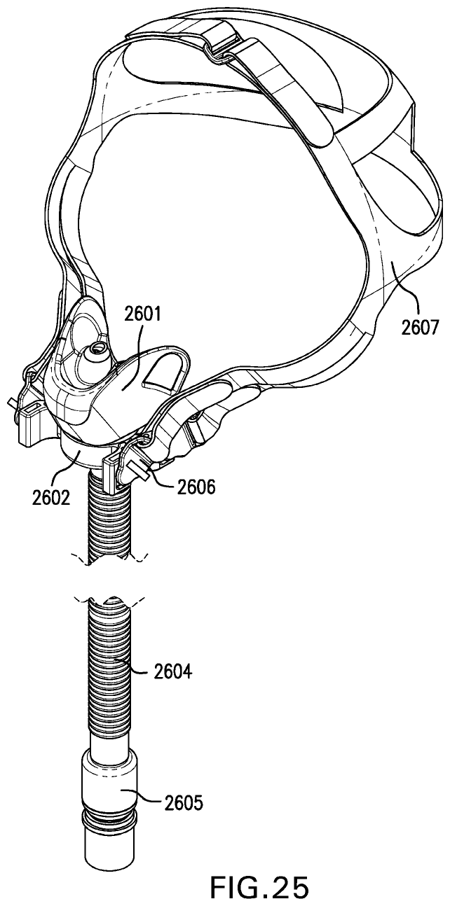

FIG. 26 depicts a resilient elastomeric chamber body component of a nasal interface system of the present invention.

FIG. 27 depicts a housing of the present invention providing an intermediate coupling means between a resilient elastomeric chamber body of the present invention and a gas supply hose.

FIG. 28 depicts a housing for coupling to a nasal interface system of the present invention configured for a 4-point retaining harness.

FIG. 29 depicts a exemplary embodiment in which exhaust apertures are provided in the substantially rigid housing instead of in the resilient elastomeric chamber body.

FIG. 30 depicts the substantially rigid housing of FIG. 28 in detail.

FIG. 31 depicts a chin-mounted CPAP device comprising dovetail rail system.

FIG. 32 depicts wall thicknesses (in mm) at various points on an exemplary resilient elastomeric nasal interface chamber body. These measurements apply to both the nasal interface as a standalone device and as a component of an oronasal interface.

### DETAILED DESCRIPTION OF THE INVENTION

The present invention provides an oronasal interface system for use with PAP systems for providing positive air pressure, particularly for use in Continuous Positive Airway Pressure (CPAP) devices, bilevel pressure support devices (BiPAP) devices and related respiratory support devices.

### Oronasal Interfaces

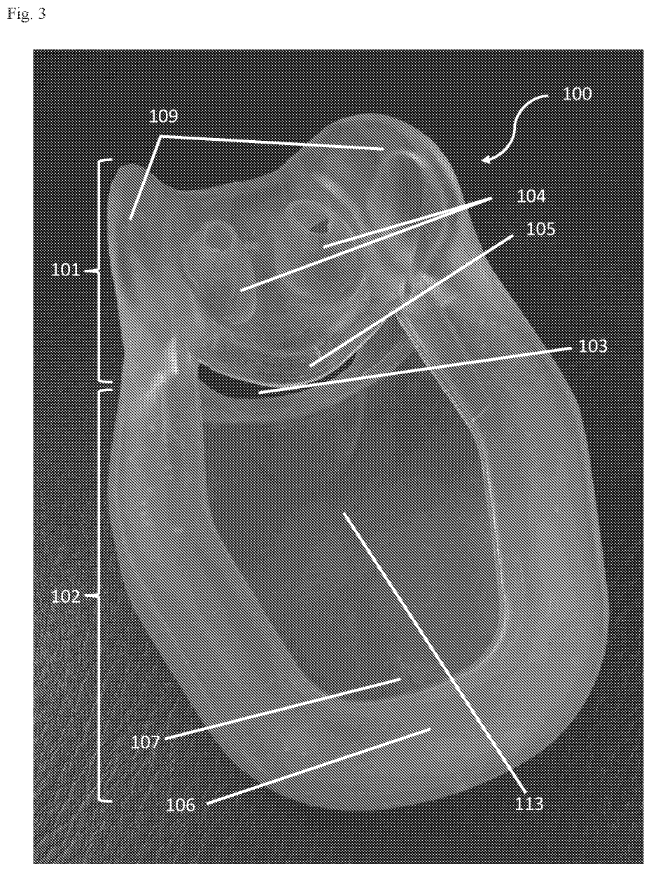

FIGS. 3, 4 and 5 depict an exemplary oronasal interface system 100. A nasal interface portion 101 is formed as a single unitary piece with an oral interface portion 102 of a resilient biocompatible elastomeric material such as a silicone elastomer, polyurethane elastomer, or polyurethane siloxane copolymer elastomer which defined an external surface and a hollow interior. A gas ingress port 103 is positioned in the rear aspect of oronasal interface system 100 and is in fluid communication with both the pair of nasal interfaces 104 and the plenum space 113 provided by the approximately bowl-shaped oral interface portion 102. The pair of nasal interfaces 104 are molded into the unitary device in the form of hollow, approximately conical, frustra that terminate in an opening fluidly connected with the hollow interior of nasal interface portion 101. The nasal interfaces 104 (e.g., prongs having an approximately frustroconical shape) fit into, and seal with, the nostrils of the wearer and have an upper opening through which pressurized gas is delivered to the nares of the user. The lower portion of oral interface portion 102 provides a chin cup 107 that embraces the mental protuberance of the wearer. A peripheral seal around the wearer's mouth is provided by the lower boundary 105 of nasal interface portion 101 which is continuous with a sealing rim 106 of oral interface portion 102.

The regionally stiffer side portions of nasal interface portion 101 wrap around the nose of the wearer, contacting the sides of the nose at the skin contact surface of the nasal interface portion 101 chamber body. This contact surface presents with a concave, bowl-shaped profile. This provides a skin contact surface that embraces the nose on either side, with the rhinal columella of the wearer positioned at approximately the bottom of the “bowl” lying superior to lower boundary 105 of nasal interface portion 101. The dorsum nasi is substantially external of the nasal interface portion 101. This results in the superior margin of the skin contact surface terminating at, or inferior to, the supratip break, and the inferior margin of the skin contact surface terminating at or superior to the upper vermillion border. The concave chamber body continues laterally from the nose with two side members, or “wings”, the inner surface of which form part of the skin contact surface of nasal interface portion 101, with an exterior surface that lies distal to that contact surface. The stiffer wings in conjunction with the immediately adjacent conical frustra gently grips the sides of the nostrils for an immediate and secure “fit” of the interface on the user's nose.

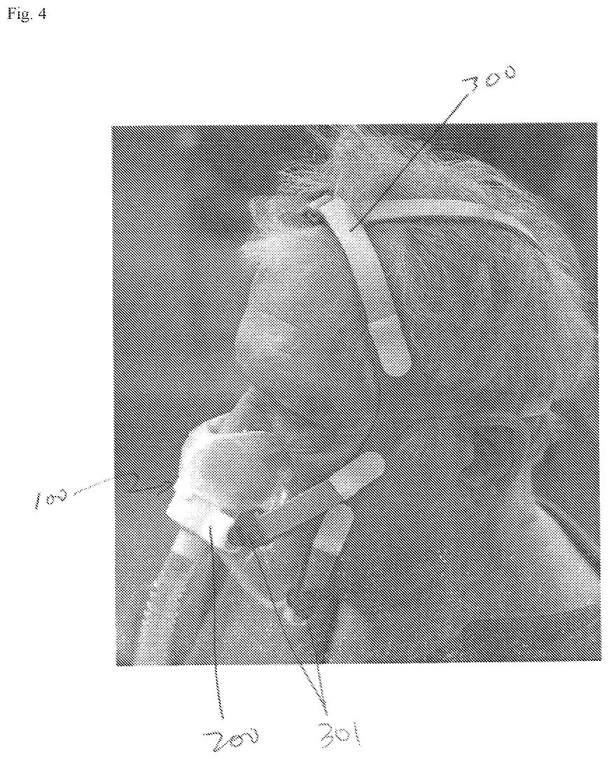

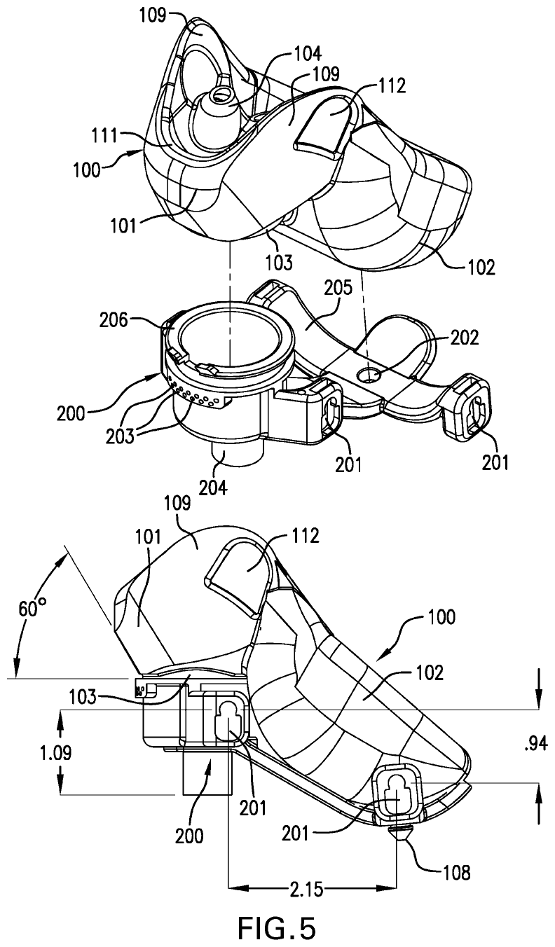

FIG. 4 shows the exemplary oronasal interface system 100 on the face of a wearer. The gas ingress port 103 in FIG. 5 is connected to base 200 which permits fluid communication of a source of pressurized gas to the nasal and oral interface portions.

Base 200 is provided as substantially rigid housing having upper surface 206 that mates and seals with gas ingress port 103 and lower surface configured to operably connect to a pressurized gas source (i.e., a PAP blower), thereby providing a fluidly connected pathway for delivery of pressurized gas to the wearer. The air pathway may optionally incorporate a hygroscopic condenser humidifier element that inserts into the base 200.

The humidifier element retains moisture when the user exhales, and this moisture is in turn returned as humidified pressurized air on inspiration.

To enhance the stability of the oronasal interface system on the wearer, a headgear 300 having four points of attachment 301 to the substantially rigid housing 200 is depicted. This headgear attaches with rotating “buttons” that “clip” into and out of mating recessed holes on the substantially rigid housing 200. It will be understood that the oronasal interface system and headgear can be larger or smaller as required to fit different facial features (e.g., nose sizes/shapes), and with different attachment means.

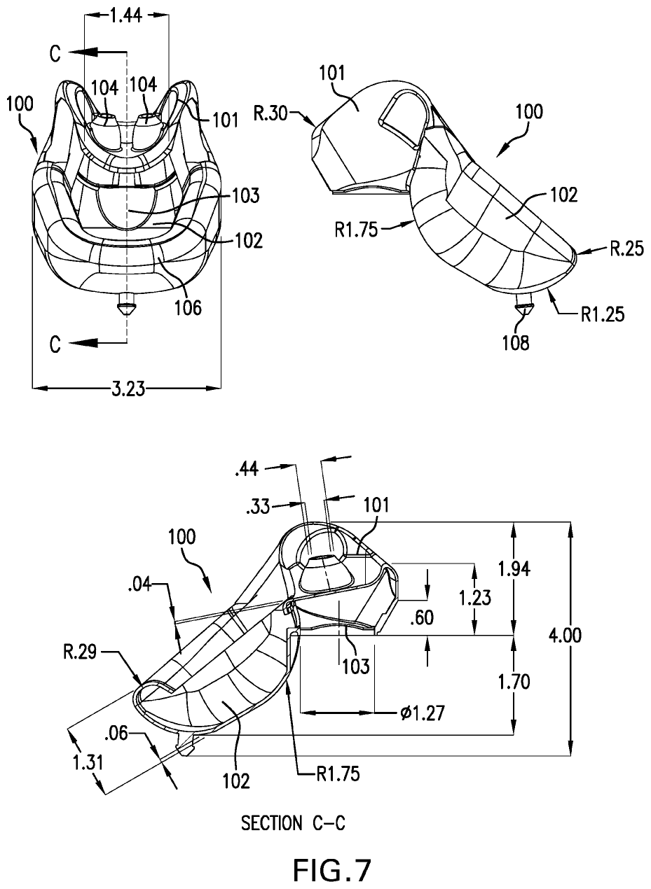

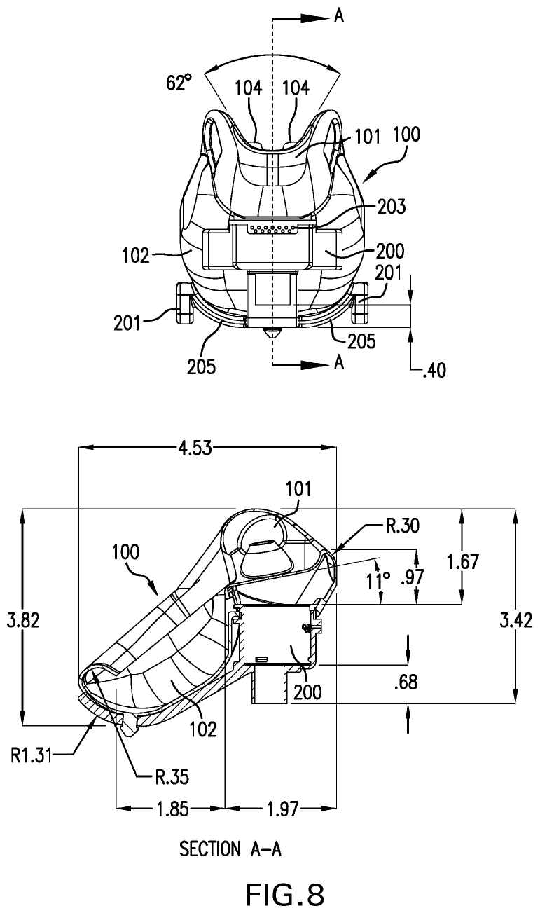

For FIGS. 5-8, all angles are in degrees, diameters φ in millimeters, and linear dimensions in inches. Gas ingress port 103 mates to the upper opening 206 of frame 200, and resilient pin 108 of the oronasal interface system 100 inserts into opening 202 of frame 200 to stabilize the frame on the oronasal interface system. Opening 202 may be located at different or multiple points on the frame and can be a groove rather than a hole to allow additional flexibility of the elastomeric cushion in the frame. Lateral arms 205 wrap around the exterior of oronasal interface system 100 and terminate in the recessed holes 201 that reversibly receive the connectors 301 on headgear 300. Coupling 204 is configured to receive an air hose or other conduit from the pressurized air supply (e.g., a CPAP blower unit). Exhaust apertures 203 in the frame each having a diameter of about 1.19 mm and a flow path length about of 5 mm, provides comparable high exhaust flows when compared to the range observed for a commercially available BREVIDA™ (Fisher and Paykel) CPAP mask at pressures between 5 and 16 cm H₂O. At the same time, this arrangement of tubular exhaust apertures is about 3 dBA quieter than the BREVIDA™ mask at 10 cm H₂O pressure. Without wishing to be bound by any theory, the use of tubular exhaust apertures is believed to smooth what is a noisy turbulent airflow to a quieter, more laminar, flow.

To provide comfort to the user as the soft section of the nasal interface portion is pushed upwards onto the user's nares, the immediate contacting surface in one embodiment is flat under the nose, with one or two outlet holes directing the airflow into the nares. While depicted as hollow, approximately conical frustra configured to insert into a nostril in these figures, the nasal interface in the nasal interface portion may be openings that do not insert into the nostril bur rather abut the nostril. Correct positioning and fit is enhanced by the addition of frustoconical nasal prongs above the outlet holes of the interface. Stability of the nasal interface is further enhanced by having a cup like structure surrounding the nasal prongs/outlet holes to envelop the base of the nose. The interface also has relatively stiffer/thicker elastomeric side members to stop sideways movement of the interface relative to the nares and cause misalignment of the outlet holes with the nares. The height of these elastomeric side members relative to the flat area under the nares is a minimum of 10 mm and a maximum extending to 10 mm above the bottom of the nasal bone. The elastomeric side members are configured to be appropriately compliant to be comfortable, but stiff enough to firmly secure the nose and not distort so as to impede movement of the interface on the wearer. The cutout on top of the soft section of the interface stops the interface touching the bony bridge of the nose, which is often irritated and eroded by the skin pressure from conventional over-the-nose CPAP masks.

The air pathway may optionally incorporate a hygroscopic condenser humidifier element that inserts into the bore of the frame or the gas ingress port 103 or as part of an optional adapter may be provided that provides a first connection to the end of flexible supply conduit and a second connection to the coupling 204. Different adapters may be produced to adapt the oronasal interface system of the present invention to a variety of manufacturer's PAP blowers.

A detailed view of an exemplary nasal interface portion 101 is described as a discrete unitary chamber in U.S. Provisional Patent Application 63/619,689, which is hereby incorporated by reference in its entirety and shown in FIGS. 5-8. The sides (“wings”) 109 of nasal interface portion 101 wrap around the nose of the wearer. The skin contact surface of the nasal interface portion 101 presents with a concave, bowl-shaped profile. This provides a skin contact surface that embraces the nose on either side, with the rhinal columella of the wearer positioned at approximately the bottom of the “bowl.” A first “superior” margin 111 of the skin contact surface is positioned proximal to the subnasale, while a second “inferior” margin 110 of the skin contact surface is proximal to the rhinal apex. The dorsum nasi is substantially external of the chamber body. A slight depression or recess may be present at the rhinal apex location to reduce pressure on the tip of the nose without compromising the peripheral seal. The shape of the skin contact surface causes the greatest pressure to overlie the bony structure of the maxilla at the position of the upper lip when the oronasal interface system 100 is properly supported on the wearer by the headgear 300.

The bowl-shaped surface continues into two side member “wings” 109, the inner surface of which form part of the skin contact surface, and comprise an exterior surface that lies distal to that contact surface. The inner skin contact surface extends over the nasal ala towards a position overlying the maxilla, and terminates skin contact proximal to a location overlying the zygomaticomaxillary suture. One or more vent holes may be positioned through this exterior surface at or near the base of wings. During expiration, exhaled gas is deposited in the hollow interior, which can exit through these pre-existing vent holes.

The distal portion of wings 109 need not be part of the hollow cavity of nasal interface portion 101, but rather the upper part of wings 109 may be solid features that remain a unitary part of nasal interface portion 101. A recess or thinned section 112 may be present in the external surface of the solid wing structure at approximately a position where the wings overlie the portion of the maxilla that is proximal to the zygomaticomaxillary suture. The location of transition between the soft lower portion of the wings and harder and relatively thicker upper portion of wings 109 creates a hinging feature, enhanced by the softer recess feature 112. This permits wings 109 to flex at a controlled location in order to allow a comfortable fit and seal on the cheeks. By this design the hollow cavity portion of nasal interface portion 101 is comfortably soft under the nose, relatively stiff at the thicker portion of the wings, and less stiff at the recesses. In addition, making at least part of the wings 109 solid reduces dead space inside nasal interface portion 101, reduces air turbulence and the resulting noise, and improves cleanability of the chamber. The thinner recessed portion also increases comfort where the wings contact the cheeks. The shape of the hollow chamber stops the interface from contacting the bony bridge of the nose, which is often irritated and eroded by the skin pressure from conventional over-the-nose CPAP masks.

Tapered nasal prongs 104 provide a more secure nasal seal when gently pushed into the nares by the interface cushion contact surface under the nose and position air outlets into the nasal cavity. They also act as gentle nasal dilators to reduce nasal resistance and minimize CPAP pressure needs. Stability and seal of the nasal interface is further enhanced by having a cup like structure surrounding the nasal prongs to envelop the base of the nose nasal interface portion 101. The cup-like structure of nasal interface portion 101 can have optional recesses molded into the contact surface at areas under the tip of the nose, over the upper lip and the sides of the nostrils to reduce “hot spot” high contact pressures at these points. The soft nasal prongs coupled with the stiffer high wings allow easy fitment of the mask and provide improved stability of the mask on the nose (e.g., when the wearer of the mask or the attachment hose moves). The chin cup of the oral interface portion 102 provides an anchor that further enhances stability of the oronasal interface system on the wearer when supported by the headgear 300.

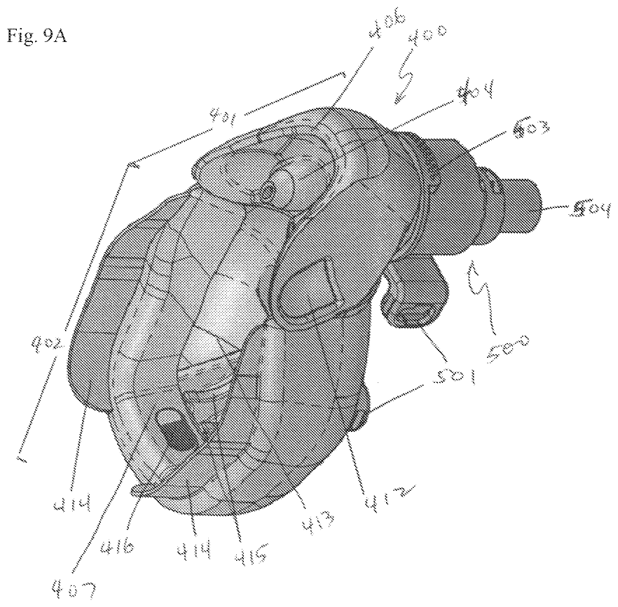

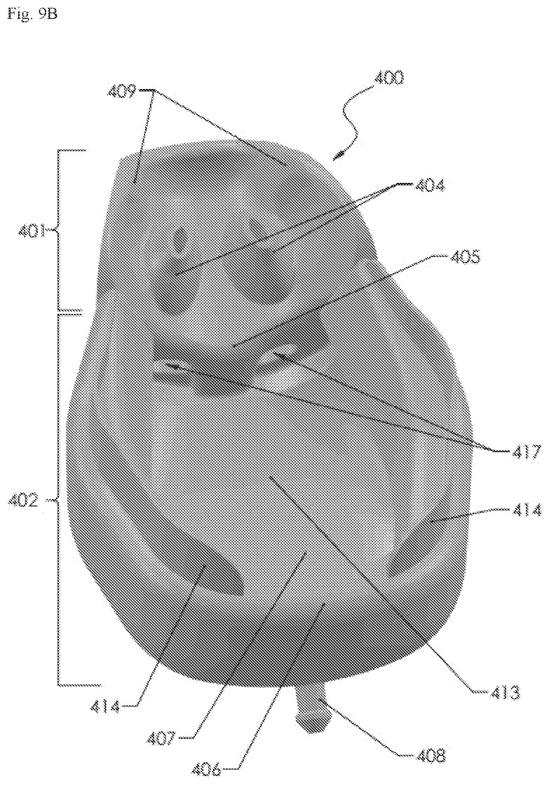

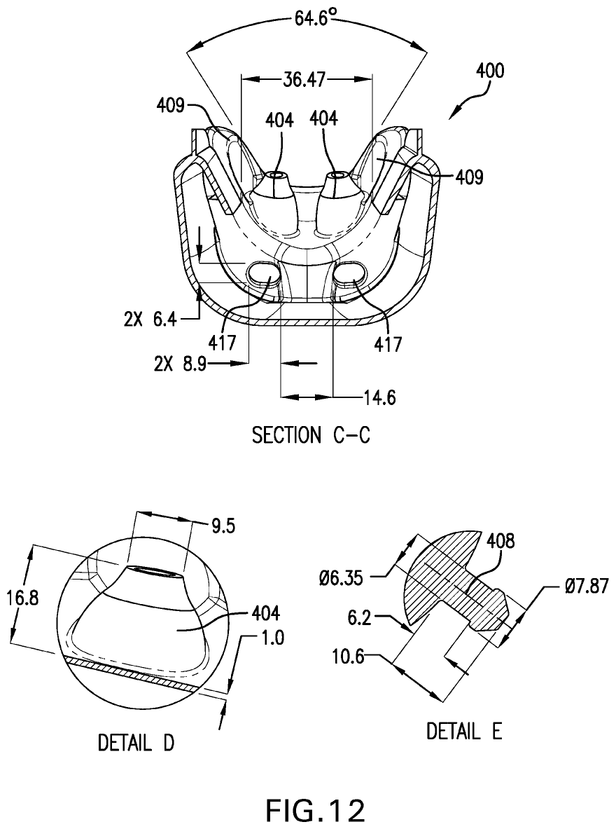

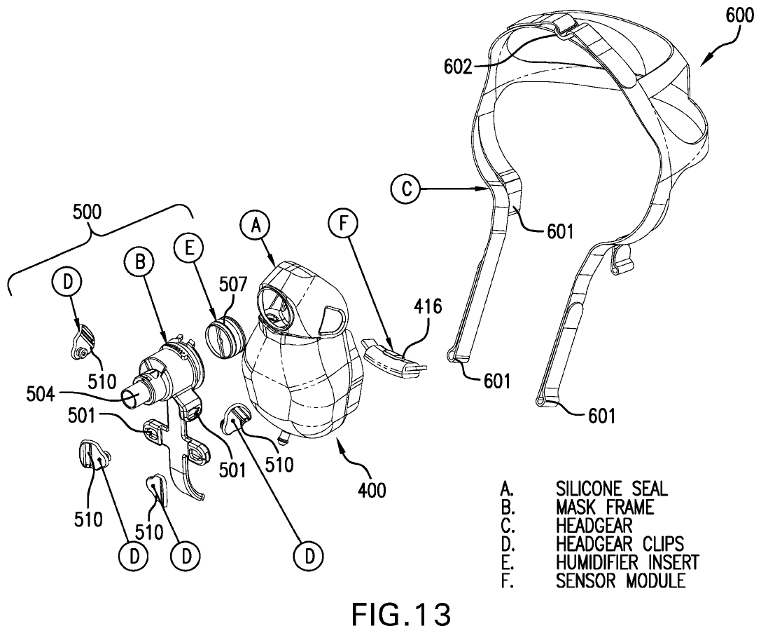

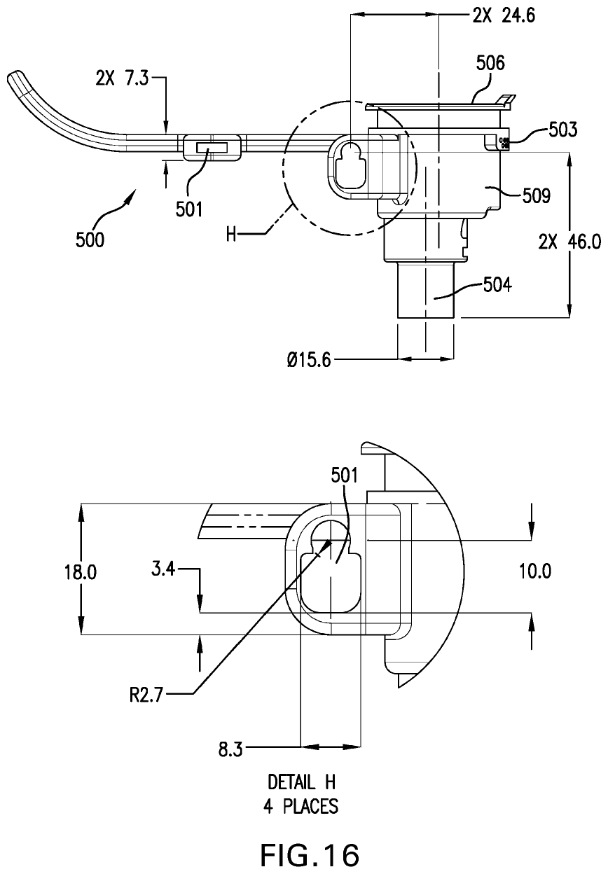

FIGS. 9-17 depict a second exemplary oronasal interface system 400 and associated frame 500. A nasal interface portion 401 is formed as a single unitary piece with an oral interface portion 402 of a resilient biocompatible elastomeric material such as a silicone elastomer, polyurethane elastomer, or polyurethane siloxane copolymer elastomer which defined an external surface and a hollow interior. A gas ingress port 403 is positioned in the rear aspect of oronasal interface system 400 and is in fluid communication with both the pair of nasal interfaces 404 and the plenum space 413 provided by the approximately bowl-shaped oral interface portion 402. The pair of nasal interfaces 404 are molded into the unitary device in the form of hollow, approximately conical, frustra that terminate in an opening fluidly connected with the hollow interior of nasal interface portion 401. The nasal interfaces 404 (e.g., approximately frustroconical prongs) fit into, and seal with, the nostrils of the wearer and have an upper opening through which pressurized gas is delivered to the nares of the user. The shape of these nasal interfaces may be altered according to a desired design, or may be omitted entirely in favor of simple openings in nasal interface portion 401 at the position of the nostrils.

The lower portion of oral interface portion 402 provides a chin cup 407 that embraces the mental protuberance of the wearer. A peripheral seal around the wearer's mouth is provided by the lower boundary 405 of nasal interface portion 401 which is continuous with a sealing rim 406 of oral interface portion 102.

Oronasal interface system 400 differs from oronasal interface system 100, in several aspects. First, resilient ribs 415 are molded into the lower end of the chin cup.

These ribs provide support for a sensor module 416 which inserts into the interior of the oral interface portion 402 at a position just under the wearer's chin. In this embodiment, the sensor module is inserted under a flap that forms the skin contact surface of chin cup 407 by sliding over and past ribs 415, which then act to hold sensor module 416 by means of a friction or snap fit design under the skin contacting flap. In designs in which the sensor module incorporates a pulse oximeter, the material forming the skin contact surface of oral interface portion 402 can be made effectively transparent to the light wavelengths utilized by the pulse oximeter. The term “effectively transparent” as used herein refers to sufficient transparency to the light wavelengths that the sensor is capable of producing a photoplethysmogram signal that is effective to measure pulse waves from the capillaries of the wearer's chin. Reliable measurements obtained from sensors mounted on the neck or the chest can be superior to those obtained from sensors on a wrist. A convenient and removable sensor module mounted on a CPAP mask allows the user to not need to wear an extra wearable device and is mounted in an optimum position for sleep quality actigraphy measurements. It also allows easy measurement of body position and breath and cardiac sounds, similar to a chest sensor, all of which have additional diagnostic value. In testing, an off the shelf pulse oximeter unit functioned through up to 4 mm thickness of silicone. Alternatively, small openings in the material forming the skin contact surface of oral interface portion 402 can be provided over the light source(s) and photodetectors of the pulse oximeter. Without limitation, other sensors can be added, such as an accelerometer for monitoring body position and sleep quality by actigraphy or a microphone for detecting breath/cardiac sounds. By encapsulating the sensor in a translucent biocompatible material, this eliminates sensor module biocompatibility issues from skin contact and being in the air delivery pathway, and this invention also protects the sensor module from drooling saliva and sweat. This invention can be applied in like fashion under the sealing flap of other mask designs.

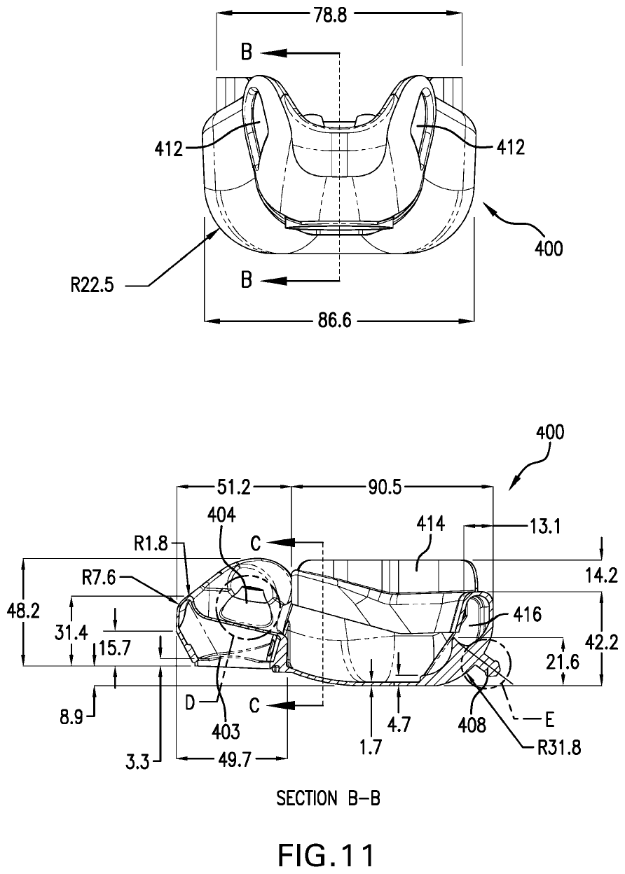

Second, oronasal interface system 400 differs from oronasal interface system 100 in that the gas ingress port 403 lies in a plane that is approximately in parallel with the long axis of oronasal interface system 400 as shown in FIG. 11, section B-B. In contrast, the gas ingress port 103 of oronasal interface system 100 lies in a plane that is approximately 45 degrees compared with the long axis of oronasal interface system 400 as shown in FIG. 5.

Third, oronasal interface system 400 comprises a pair of resilient flaps 414 which extends down each side external to the plenum space and configured to lie on the cheeks (buccæ) on the sides of the face to provide additional stability and improved seal when the oronasal interface system 400 is positioned on the wearer. In an adult size oronasal interface, these resilient flaps 414 may extend a minimum of 50 mm, although this length will vary depending on the size of the oronasal interface.

The sides of nasal interface portion 401 wrap around the nose of the wearer, contacting the sides of the nose at the skin contact surface of the nasal interface portion 401 chamber body. This contact surface presents with a concave, bowl-shaped profile.

This provides a skin contact surface that embraces the nose on either side, with the rhinal columella of the wearer positioned at approximately the bottom of the “bowl” lying superior to lower boundary 405 of nasal interface portion 401. The dorsum nasi is substantially external of the nasal interface portion 401. This results in the superior margin of the skin contact surface terminating at, or inferior to, the supratip break, and the inferior margin of the skin contact surface terminating at or superior to the upper vermillion border. The concave chamber body continues laterally from the nose with two side members, or “wings” 412, the inner surface of which form part of the skin contact surface of nasal interface portion 401, with an exterior surface that lies distal to that contact surface. The relatively stiffer wings gently hold the sides of the nostrils between the wings and the approximately frustroconical prongs.

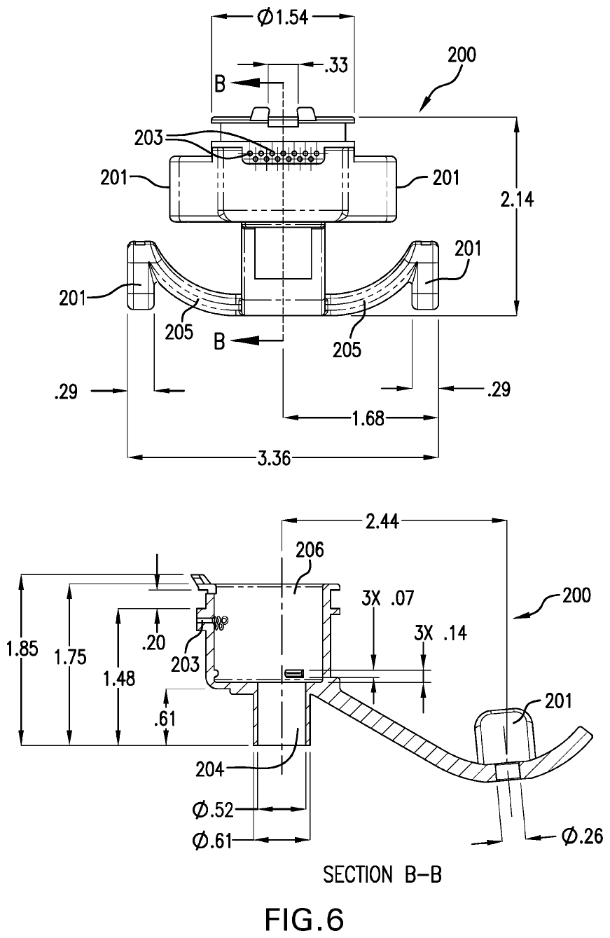

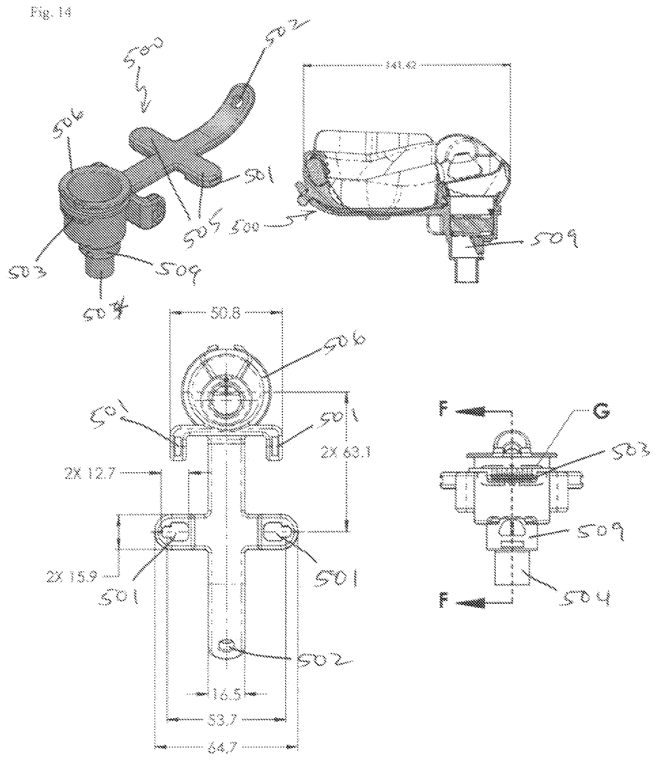

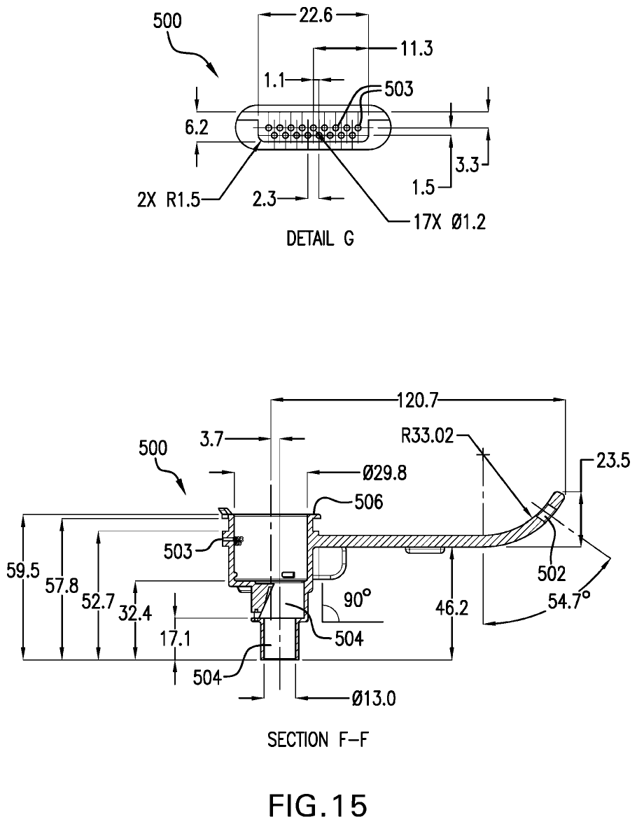

Base (or “frame”) 500 is shown in FIGS. 14-17. The base is provided as a substantially rigid housing having upper surface 506 that mates and seals with gas ingress port 503 and lower surface configured to operably connect to a pressurized gas source (i.e., a PAP blower), thereby providing a fluidly connected pathway for delivery of pressurized gas to the wearer. The air pathway may optionally incorporate a hygroscopic condenser humidifier element 507 that inserts into the base 500. The humidifier element retains moisture when the user exhales, and this moisture is in turn returned as humidified pressurized air on inspiration. In keeping with the planar positioning of the gas ingress port 406 relative to the long axis of oronasal interface system 400, base 500 is more linear in profile as shown in Section F-F of FIGS. 15-17. That is, the long axis of the air path from air hose coupling 504 to upper opening 506 is at approximately 90° to the long axis of frame 500. This is as compared to base 200 shown in FIG. 6, in which the long axis of the air path from air hose coupling 204 to upper opening 206 is at approximately 45° to the long axis of frame 200.

To enhance the stability of the oronasal interface system on the wearer, a headgear 600 having four points of attachment 601 to the substantially rigid frame 500 is depicted. This headgear attaches with rotating “buttons” 510 that “clip” into and out of mating recessed holes 501 on the substantially rigid frame 500. It will be understood that the oronasal interface system and headgear can be larger or smaller as required to fit different facial features (e.g., nose sizes/shapes), and with different attachment means.

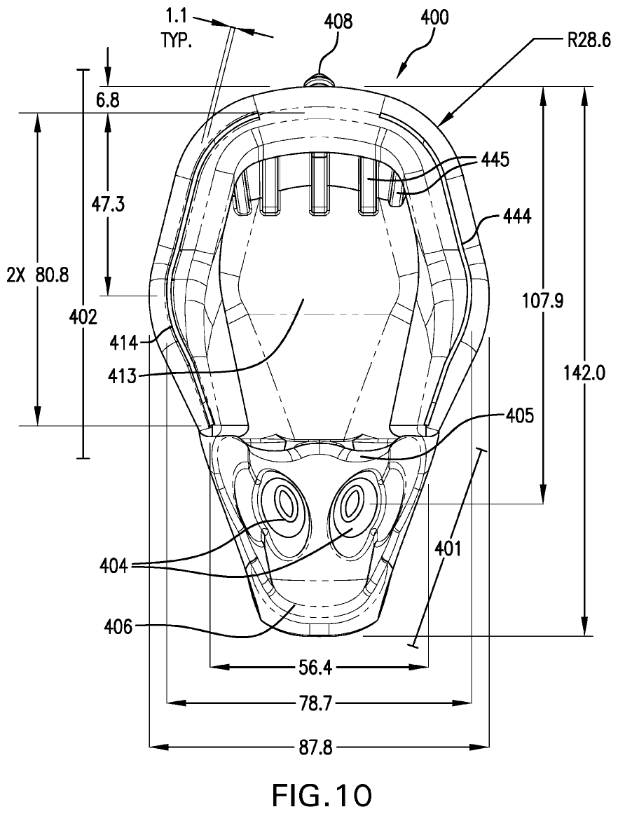

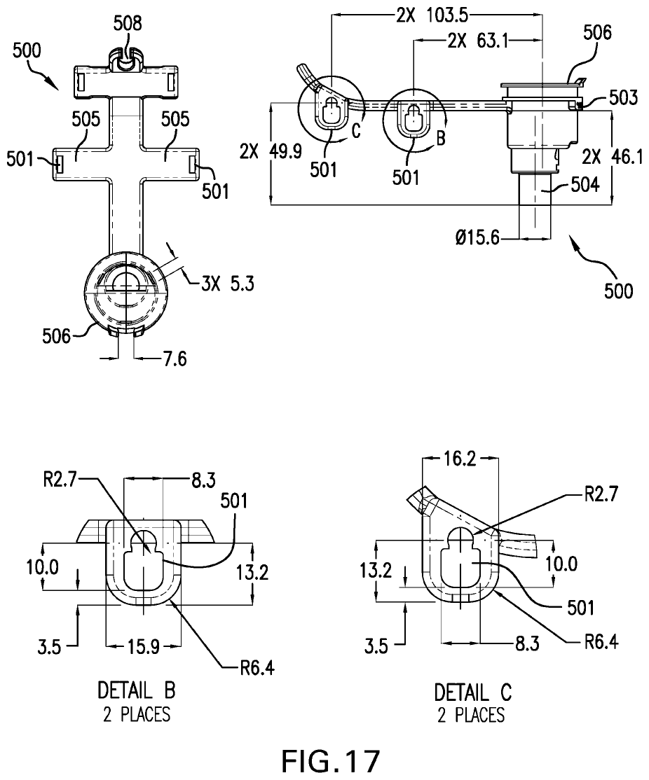

For FIGS. 10-17, all dimensions shown are in mm. Gas ingress port 403 mates to the upper opening 506 of frame 500, and resilient pin 408 of the oronasal interface system 400 inserts into opening 502 (FIG. 14) or slot 508 (FIG. 17) of frame 500 to stabilize the frame on the oronasal interface system. Lateral arms 505 wrap around the exterior of oronasal interface system 400 and terminate in the recessed holes 501 that reversibly receive the connectors 601 on headgear 600 via the connecting button clips 510. Coupling 504 is configured to receive an air hose or other conduit from the pressurized air supply (e.g., a CPAP blower unit). Exhaust apertures 503 are provided in the frame, each having a diameter of about 1.19 mm and a flow path length about of 5 mm. FIGS. 14-17 also depict a location in the frame for an anti-asphyxiation valve 509 that, by opening to atmosphere in a failsafe manner, reduces the risk of excessive CO₂ rebreathing by a patient.

To provide comfort to the user as the soft section of the nasal interface portion 401 is pushed upwards onto the user's nares, the immediate contacting surface in one embodiment is flat under the nose, with one or two outlet holes directing the airflow into the nares. While depicted as hollow, approximately conical frustra configured to insert into a nostril in these figures, the nasal interface in the nasal interface portion may be openings that do not insert into the nostril bur rather abut the nostril. Correct positioning and fit are enhanced by the addition of frustoconical nasal prongs above the outlet holes of the interface. Stability of the nasal interface is further enhanced by having a cup like structure surrounding the nasal prongs/outlet holes to envelop the base of the nose. The interface also has elastomeric side members to stop sideways movement of the interface relative to the nares and cause misalignment of the outlet holes with the nares. The height of these elastomeric side members relative to the flat area under the nares is a minimum of 10 mm and a maximum extending to 10 mm above the bottom of the nasal bone. The elastomeric side members are configured to be appropriately compliant to be comfortable. The cutout on top of the soft section of the interface stops the interface touching the bony bridge of the nose, which is often irritated and eroded by the skin pressure from conventional over-the-nose CPAP masks.

The air pathway may optionally incorporate a hygroscopic condenser humidifier element 507 that inserts into the bore of the frame or the gas ingress port 403 or as part of an optional adapter may be provided that provides a first connection to the end of flexible supply conduit and a second connection to the coupling 504. Different adapters may be produced to adapt the oronasal interface system of the present invention to a variety of manufacturer's PAP blowers.

A detailed view of an exemplary nasal interface portion 401 is described hereinafter. The sides (“wings”) 409 of nasal interface portion 401 wrap around the nose of the wearer. The skin contact surface of the nasal interface portion 401 presents with a concave, bowl-shaped profile. This provides a skin contact surface that embraces the nose on either side, with the rhinal columella of the wearer positioned at approximately the bottom of the “bowl.” A first “superior” margin of the skin contact surface is positioned proximal to the subnasale, while a second “inferior” margin of the skin contact surface is proximal to the rhinal apex. The dorsum nasi is substantially external of the chamber body. A slight depression or recess may be present at the rhinal apex location to reduce pressure on the tip of the nose without compromising the peripheral seal. The shape of the skin contact surface causes the greatest pressure to overlie the bony structure of the maxilla at the position of the upper lip when the oronasal interface system 400 is properly supported on the wearer by the headgear 600. The inner skin contact surface extends over the nasal ala towards a position overlying the maxilla, and terminates skin contact proximal to a location overlying the zygomaticomaxillary suture. One or more vent holes may be positioned through this exterior surface at or near the base of wings. During expiration, exhaled gas is deposited in the hollow interior, which can exit through these pre-existing vent holes.

The distal portion of wings 409 need not be part of the hollow cavity of nasal interface portion 401, but rather the upper part of wings 409 are solid features that remain a unitary part of nasal interface portion 401. A recess or thinned section may be present in the external surface of the solid wing structure at approximately a position where the wings overlie the portion of the maxilla that is proximal to the zygomaticomaxillary suture. The location of transition between the soft lower portion of the wings and harder and relatively thicker upper portion of wings 409 creates a hinging feature, enhanced by the softer recess feature. This permits wings 409 to flex at a controlled location in order to allow a comfortable fit and seal on the cheeks. By this design the hollow cavity portion of nasal interface portion 401 is comfortably soft under the nose, relatively stiff at the thicker portion of the wings, and less stiff at the recesses. In addition, making at least part of the wings 409 solid reduces dead space inside nasal interface portion 401, reduces air turbulence and the resulting noise, and improves cleanability of the chamber. The thinner recessed portion also increases comfort where the wings contact the cheeks. Also the shape of the hollow chamber stops the interface from contacting the bony bridge of the nose, which is often irritated and eroded by the skin pressure from conventional over-the-nose CPAP masks. Openings 417 fluidly connect the hollow cavity portion of nasal interface portion 401, the plenum space of oronasal interface system 400, and upper opening 506 of frame 500.

Tapered nasal prongs 404 provide a more secure nasal seal when gently pushed into the nares by the interface cushion contact surface under the nose and position air outlets into the nasal cavity. They also act as gentle nasal dilators to reduce nasal resistance and minimize CPAP pressure needs. Stability and seal of the nasal interface is further enhanced by having a cup like structure surrounding the nasal prongs to envelop the base of the nose nasal interface portion 401. The cup-like structure of nasal interface portion 401 can have optional recesses molded into the contact surface at areas under the tip of the nose, over the upper lip and the sides of the nostrils to reduce “hot spot” high contact pressures at these points. The soft nasal prongs coupled with the stiffer high wings gently embrace the sides of the nostrils allowing easy fitment of the mask and provide improved stability of the mask on the nose (e.g., when the wearer of the mask or the attachment hose moves). The chin cup of the oral interface portion 402 provides an anchor that further enhances stability of the oronasal interface system on the wearer when supported by the headgear 600.

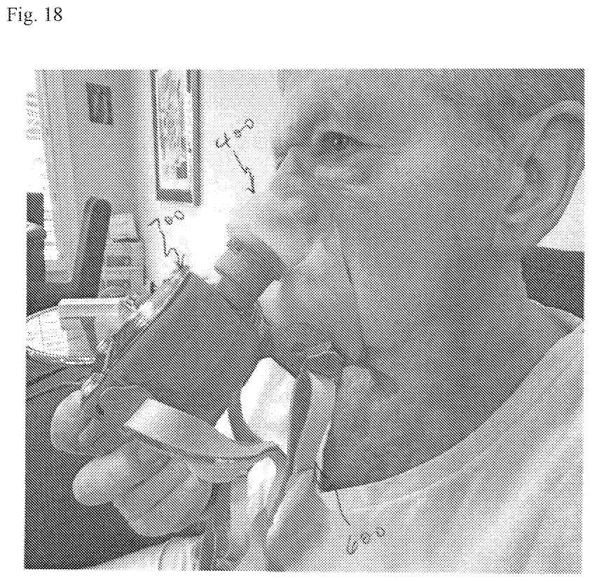

In addition to those aspects described herein which comprise the use of a frame, FIG. 18 depicts the use of an oronasal interface of the present invention in which the “frame” is provided in the form of an integrated PAP blower such as that described in PCT/US2022/043556, which is hereby incorporated by reference.

Nasal Interfaces The present invention also provides a nasal interface system for use with PAP systems for providing positive air pressure, particularly for use in Continuous Positive Airway Pressure (CPAP) devices, bilevel pressure support devices (BiPAP) devices and related respiratory support devices.

Conventional PAP interfaces often have air paths which add turbulence and noise to the gas flowing through them. The nasal interface described herein provides a substantially straight pathway from the CPAP unit outlet, through a bulbous resonant chamber, to the interface's gas outlet at the user's nares. Such a design has minimal abrupt geometry changes in the airflow path to minimize turbulence and noise, and so can provide less disruptive noise than some conventional interfaces. Similarly, the design provides for a substantially linear pathway for incorporating an inline hygroscopic condenser humidifier element.

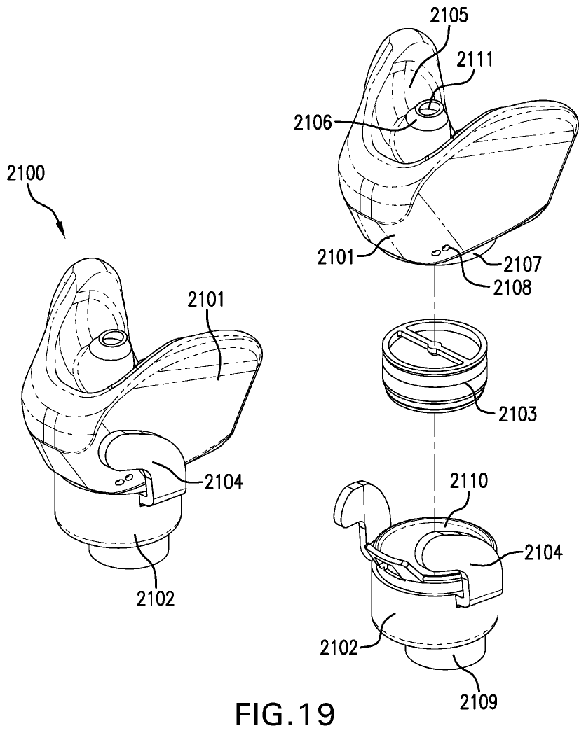

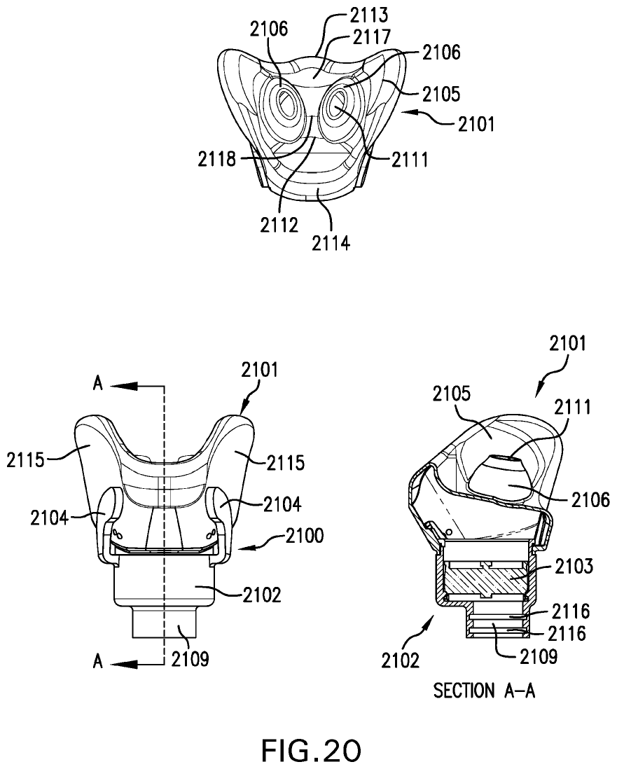

FIG. 19 depicts an exemplary nasal interface system 2100 assembled (left) and in exploded form (right). FIG. 20 depicts the resilient elastomeric chamber body in more detail from several angles. A unitary chamber 2101 is formed as a single piece of a resilient biocompatible elastomeric material such as a silicone elastomer, polyurethane elastomer, or polyurethane siloxane copolymer elastomer which defined an external surface and a hollow interior. A pair of nasal interfaces 2106 are molded into the unitary chamber in the form of hollow, approximately conical, frustra that terminate in an opening fluidly connected with the hollow interior. The interfaces 2106 (e.g., approximately frustroconical prongs) fit into, and seal with, the nostrils of the wearer and have an upper opening 2111 through which pressurized gas is delivered to the nares of the user.

The sides of unitary chamber 2101 wrap around the nose of the wearer, contacting the sides of the nose at the skin contact surface 2105 of the chamber body.

This contact surface presents with a concave, bowl-shaped profile. This provides a skin contact surface that embraces the nose on either side, with the rhinal columella of the wearer positioned at approximately the bottom of the “bowl” at location 2118. A first “superior” margin 2114 of the skin contact surface is positioned proximal to the subnasale, while a second “inferior” margin 2113 of the skin contact surface is proximal to the rhinal apex. The dorsum nasi is substantially external of the chamber body. This results in the superior margin of the skin contact surface terminating at, or inferior to, the supratip break, and the inferior margin of the skin contact surface terminating at or superior to the upper vermillion border. A surface recess 2117 in the first surface region is positioned inferior to the upper margin and configured to receive the individual's tip lobule.

The concave chamber body continues laterally from the nose with two side members, or “wings,” 2115, the inner surface of which form part of the skin contact surface, with an exterior surface that lies distal to that contact surface. Depicted in FIGS. 19-21 as providing an internal chamber volume continuous with the chamber volume of the hollow chamber body, these wings may alternatively be solid or partially solid at their upper region and so not a part of the chamber body. One or more exhaust apertures (or “vent holes”) 2108 are positioned through this exterior surface at or near the base of wings 2115. During expiration, exhaled gas is deposited in the hollow interior, which can exit through these pre-existing vent holes.

The base surface 2107 of the chamber body that is distal to the skin contact surface provides an open gas ingress port, which provides access of pressurized gas to the hollow chamber interior. A substantially rigid housing 2102 comprises an upper surface 2110 that mates and seals with gas ingress port to form the nasal interface system. A lower surface 2109 of substantially rigid housing 2102 is configured to operably connect to a pressurized gas source (i.e., a PAP blower), thereby providing a fluidly connected pathway between the pressurized gas source and the interior of the hollow chamber body 2101. O-rings 2116 can be provided on an interior or exterior surface of lower surface 2109 to provide an airtight friction fit to the pressurized gas source.

In the case where the wings 2115 of the chamber body 2101 are hollow, they may be collapsed if the wearer's head applies pressure to the exterior as might occur during sleep, compromising the fit and seal of the nasal interface system and collapse the underlying nostril. To provide some protection against collapse, the unitary substantially rigid housing 2102 can provide optional shielding portions 2104 extending across at least a portion of the external surface of each wing structure 2115. Alternatively, or together with the shielding portions, regional stiffening of the upper part of the wings can provide additional protection of the chamber body to prevent distortion and movement of the nasal interface or collapse.

The air pathway may optionally incorporate a hygroscopic condenser humidifier element 2103 that inserts into the substantially rigid housing 2102 or the base of the chamber body 2101. The humidifier element 2103 retains moisture when the user exhales, and this moisture is in turn returned as humidified pressurized air on inspiration. Moisture retention by the element on exhalation is enhanced by providing the exhaust apertures 2108 on the outside of the chamber body 2101 pointing downwards and away from the user. As described hereinafter, these exhaust apertures may also be placed elsewhere within the air pathway, such as within the substantially rigid housing.

To provide comfort to the user as the soft section of the interface is pushed upwards onto the user's nares, the immediate contacting surface in one embodiment is flat under the nose, with one or two outlet holes directing the airflow into the nares. Correct positioning and fit are enhanced by the addition of frustoconical nasal prongs above the outlet holes of the interface. Stability of the nasal interface is further enhanced by having a cup like structure surrounding the nasal prongs to envelop the base of the nose. The interface also has elastomeric side members 2115 to stop sideways movement of the interface relative to the nares and cause misalignment of the outlet holes with the nares.

The height of these elastomeric side members relative to the flat area under the nares is a minimum of 10 mm and a maximum extending to 10 mm above the bottom of the nasal bone. The elastomeric side members are configured to be appropriately compliant to be comfortable. The elastomeric side members and the entire thin-walled soft upper section of the interface may be configured to inflate slightly under the applied CPAP pressure and essentially float on the nose without significant pressure points. To avoid a side bolster and the user's nostril being crushed when the user sleeps on their side, the hard plastic base of the interface unit may include hard plastic wings protruding upwards from the base connector to protect the outside of the side bolsters. Also, the cutout on top of the soft section of the interface stops the interface touching the bony bridge of the nose, which is often irritated and eroded by the skin pressure from conventional over-the-nose CPAP masks.

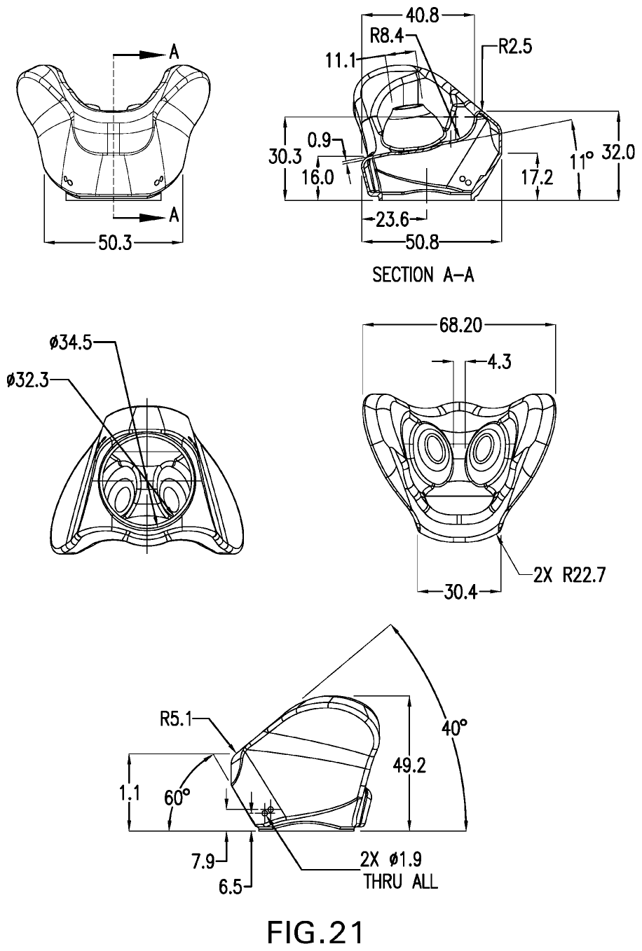

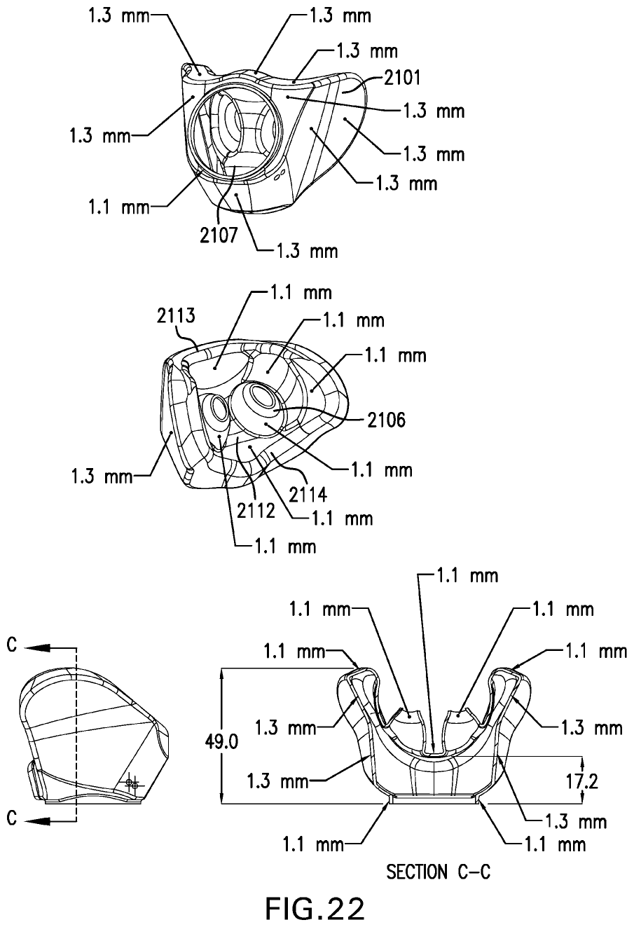

FIG. 21 depicts one example of a resilient elastomeric chamber body, showing the angles (in degrees), diameters φ (in millimeters), and linear dimensions (in millimeters) of features in this exemplary embodiment. It can be recognized that the interface design can be larger or smaller as required to fit different nose sizes/shapes and accommodate different distances between the CPAP unit and a user's nose. As well as dissipating noise, the large bulbous noise absorbing chamber acts as a compliance buffer between the nose and the hard plastic base of the interface and to avoid the user's nose contacting this hard surface. To ensure comfort, fit, seal and compliance, the soft bulbous section of the interface is preferably made from a 30 to 70 durometer biocompatible silicone (ideally 40 to 60) but higher and lower durometers and different materials may also be substituted. Exemplary wall thicknesses for various features of the resilient elastomeric chamber body are depicted in FIG. 22. The portion of the chamber body forming the space-filled interior chamber may have a Shore A durometer of between 30 and 60 and a wall thickness of between 0.5 mm and 5.0 mm, with regional differences in wall thickness (and so stiffness) used to provide regional variations in deformability.

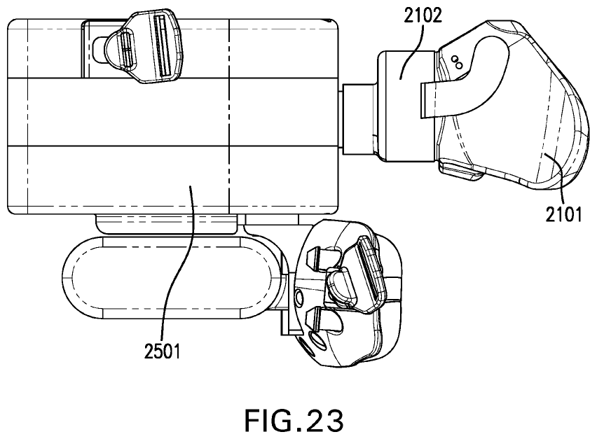

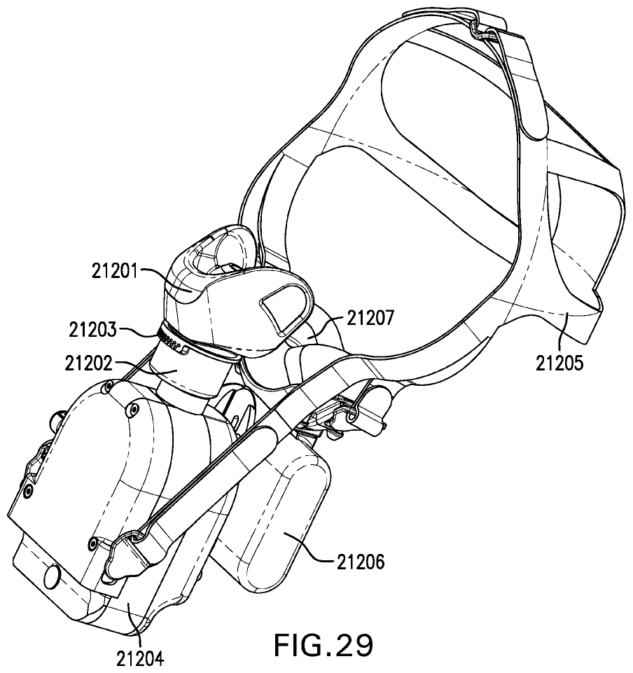

The nasal interface system of the present invention finds particularly advantageous use with the body-worn positive airway pressure devices as described in PCT/US2022/043556, in which the PAP blower unit is chin mounted and held in place by a head strap, and a face mask (e.g., nasal pillow) is positioned at the wearer's nose as an integral component of the PAP device. This use is depicted in FIGS. 23 and 29, in which unitary chamber 2101 is mounted to the unitary substantially rigid housing 2102, which is then directly coupled to the body-worn positive airway pressure device 2501. In configurations where the nasal interface system of the present invention is connected to a PAP blower unit via tubing or other means, and the nasal interface system of the present invention may itself be held in position using a head strap.

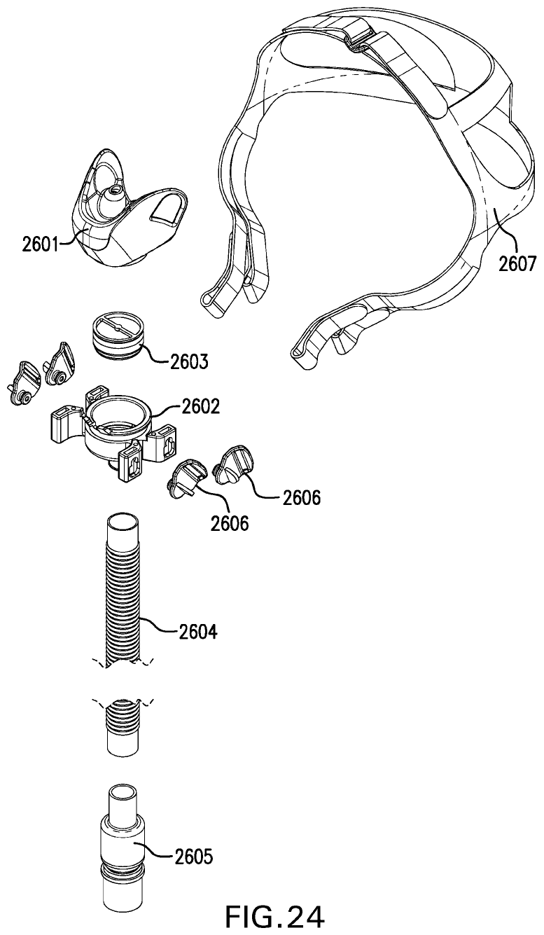

Alternatively, the nasal interface system of the present invention finds use with more traditional positive airway pressure devices that are connected to a PAP blower by a flexible supply conduit (e.g., a hose). Such an exemplary embodiment is depicted in FIG. 24 as an exploded diagram of component parts and in FIG. 24 in assembled form.

While not limited to traditional PAP blower formats, the unitary chamber 2601 is in this case is formed as a single piece of a resilient biocompatible elastomeric material such as a silicone elastomer, polyurethane elastomer, or polyurethane siloxane copolymer elastomer which defined an external surface and a hollow interior. A substantially rigid housing 2602 comprises an upper surface that mates and seals with a gas ingress port to of unitary chamber 2601, and a lower surface of substantially rigid housing 2602 is configured to operably connect to a pressurized gas source (i.e., a PAP blower) via flexible supply conduit 2604, thereby providing a fluidly connected pathway between the pressurized gas source and the interior of the hollow chamber body 2601.

The air pathway may optionally incorporate a hygroscopic condenser humidifier element 2603 that inserts into the substantially rigid housing 602 or the base of the chamber body 2601. An optional adapter 2605 may be provided that provides a first connection to the end of flexible supply conduit 2604 and a second connection that is adapted to connect the system to the output port of a particular PAP blower. Different adapters 2605 may be produced to adapt the nasal interface system of the present invention to a variety of manufacturer's PAP blowers.

To enhance the stability of the nasal interface system on the wearer, a headgear having four points of attachment to the substantially rigid housing 2602 is depicted. This headgear attaches with rotating “buttons” 2606 that “clip” into and out of mating recessed holes on the substantially rigid housing 2602. A detailed view of the substantially rigid housing 2602 is shown in FIG. 29, with the mating holes 21001 depicted as extensions from the generally circular body 21003. A coupling 21002 is provided for attachment of the flexible supply conduit. It will be understood that the interface design and headgear can be larger or smaller as required to fit different facial features (e.g., nose sizes/shapes). The nasal and oronasal system headgear designs are similar in their approach-to provide a stable 4 point attachment which pulls slightly upwards to maintain a seal under the nose, and under the chin in the case of the oronasal system. The headgear is preferably designed to sit above the ears and be adjusted for different size heads by a single strap adjustment at the top of the head. The adjustable side straps allow a gentle stable pull on the sides of the masks. The headgear may have stiffening elements, which can range from providing thicker material or sections, and/or plastic inserts in the headgear material. AN extended loop and hook closure on the top of the head can also provide a stiffer headgear. A stiffer headgear would maintain its head shape and so simplify fitting on a user.

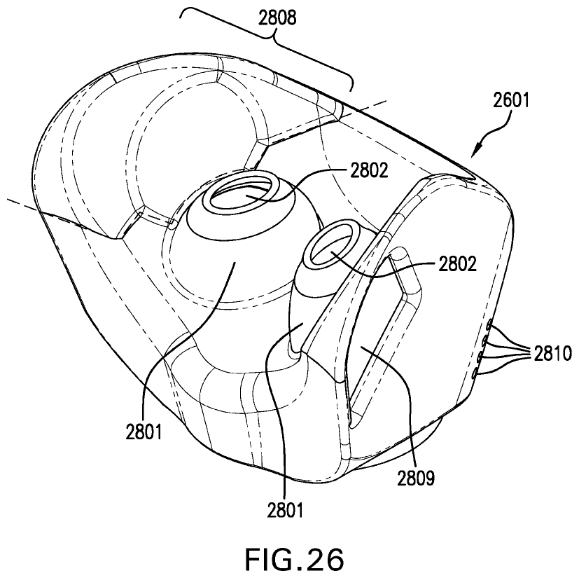

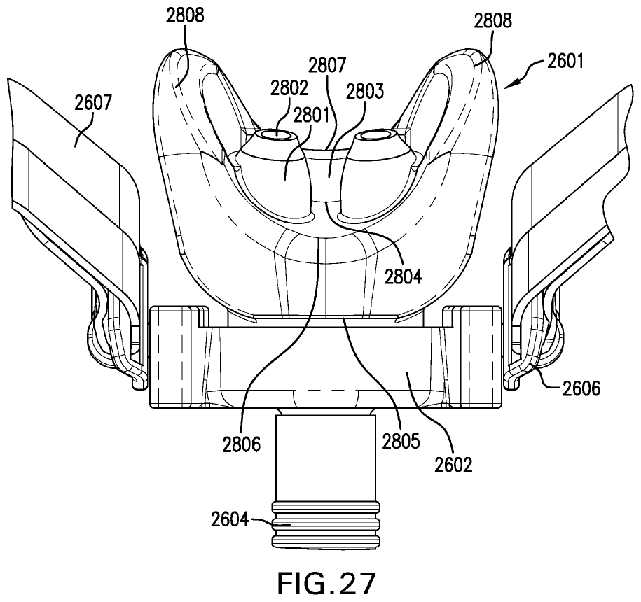

A detailed view of the unitary chamber 2601 is depicted in FIGS. 26 and 27.

The sides 2808 of unitary chamber 2601 wrap around the nose of the wearer. The skin contact surface 2803 of the chamber body presents with a concave, bowls-shaped profile. This provides a skin contact surface that embraces the nose on either side, with the rhinal columella of the wearer positioned at approximately the bottom of the “bowl.” A first “superior” margin 2807 of the skin contact surface is positioned proximal to the subnasale, while a second “inferior” margin 2806 of the skin contact surface is proximal to the rhinal apex. The dorsum nasi is substantially external of the chamber body. A slight depression or recess may be present at the rhinal apex location 2803 to reduce pressure on the tip of the nose without compromising the peripheral seal. The shape of the skin contact surface causes the greatest pressure to overlie the bony structure of the maxilla at the position of the upper lip when the unitary chamber 2601 is properly supported by the headgear 2607.

The bowl-shaped surface continues into two side member “wings” 2808, the inner surface of which form part of the skin contact surface, and comprise an exterior surface that lies distal to that contact surface. The inner skin contact surface extends over the nasal ala towards a position overlying the maxilla, and terminates skin contact proximal to a location overlying the zygomaticomaxillary suture. One or more vent holes 2810 are positioned through this exterior surface at or near the base of wings 2808.

During expiration, exhaled gas is deposited in the hollow interior, which can exit through these pre-existing vent holes.

The base, or posterior surface, 2805 of the chamber body provides an open gas ingress port, which provides access of pressurized gas to the hollow chamber interior. A substantially rigid housing 2602 comprises an upper surface that mates and seals with gas ingress port to form the nasal interface system.

In the embodiment depicted in FIGS. 26 and 27, the distal portion of wings 2808 are not part of the hollow cavity of unitary chamber 2601, but rather the upper part of wings 2808 are solid features that remain a unitary part of unitary chamber 2601. This can permit the deletion of shielding portions on rigid housing 2602 that were depicted in the embodiment of FIG. 18. A recess or thinned section 2809 may be present in the external surface of the solid wing structure at approximately a position where the wings overlie the portion of the maxilla that is proximal to the zygomaticomaxillary suture. The location of transition between the soft lower portion of the wings and harder and relatively thicker upper portion of wings 2808 creates a hinging feature, enhanced by the softer recess feature. This permits wings 2808 to flex at a controlled location in order to allow a comfortable fit and seal on the cheeks. By this design the hollow cavity portion of unitary chamber 2601 is comfortably soft under the nose, relatively stiff at the thicker portion of the wings, and less stiff at the recesses. In addition, making at least part of the wings 2808 solid reduces dead space inside unitary chamber 2601, reduces air turbulence and the resulting noise, and improves cleanability of the chamber. The thinner recessed portion also increases comfort where the wings contact the cheeks. Also, the shape of the hollow chamber reduces the propensity of the interface from contacting the bony bridge of the nose, which is often irritated and eroded by the skin pressure from conventional over-the-nose CPAP masks.

Tapered nasal prongs 2801 provide a secure nasal seal when gently pushed into the nares by the interface cushion contact surface under the nose and position air outlets 2802 into the nasal cavity. They also act as gentle nasal dilators to reduce nasal resistance and minimize CPAP pressure needs. Stability and seal of the nasal interface is further enhanced by having a cup like structure surrounding the nasal prongs to envelop the base of the nose. This cup-like structure can have optional recesses molded into the contact surface at areas under the tip of the nose, over the upper lip and the sides of the nostrils to reduce “hot spot” high contact pressures at these points. The soft nasal prongs coupled with the stiffer high wings allow easy fitment of the mask and provide improved stability of the mask on the nose (e.g., when the wearer of the mask or the attachment hose moves).

FIG. 29 depicts another exemplary embodiment in which the resilient elastomeric chamber body 21201 is operably connected to substantially rigid housing 21202. In this case, the exhaust apertures 21203 are provided in the substantially rigid housing 21202 instead of in the resilient elastomeric chamber body. This array of exhaust apertures is tubular in nature, with an open bore diameter of each of the exhaust apertures being about 1.2 mm, and the flow path length of each of the exhaust apertures is about 5 mm. A lower surface of substantially rigid housing 21202 is configured to operably connect to a pressurized gas source 21204 (i.e., a PAP blower) that is powered by a battery 21206. A headgear 21205 is used to position the device on the wearer, with the weight of the system applied to the wearer through chin cup 21207. The exhaust holes can be oriented in a variety of different directions, e.g. so as not to direct the exhaust airflow onto the user or a bed partner.

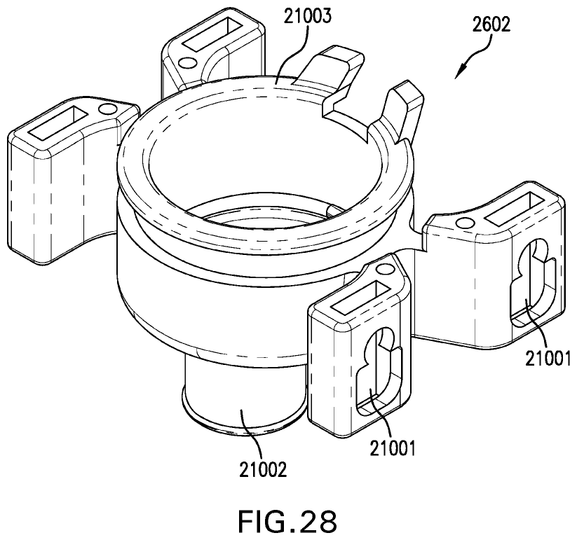

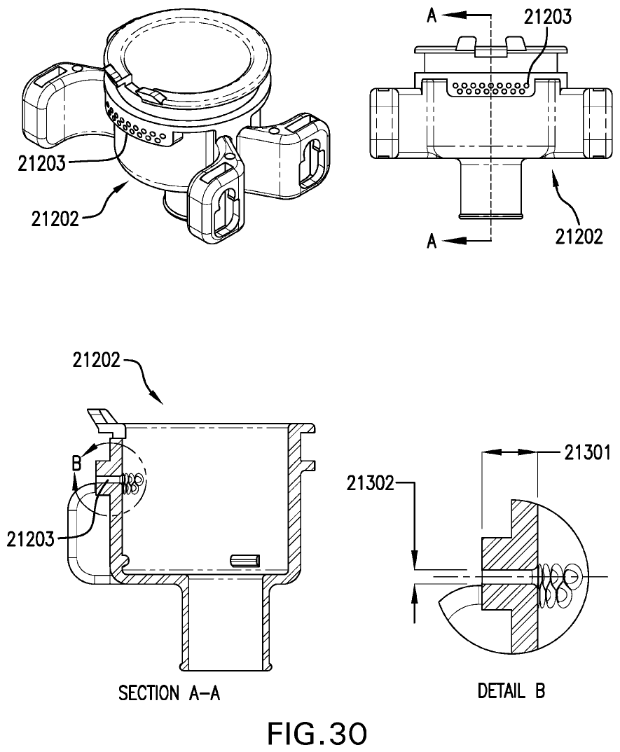

FIGS. 28 and 30 depict substantially rigid housing 21202 in more detail.

Relocating the mask exhaust holes 21203 from the resilient elastomeric chamber body to the housing can reduce the noise level during use of the nasal interface to deliver positive pressure gases at therapeutic levels of airflow into the mask. As depicted, 17 exhaust apertures 21203, each having a diameter 21302 of 1.19 mm and a flow path length 21301 of 5 mm, provides comparable high exhaust flows when compared to the range shown for a commercially available BREVIDA™ (Fisher and Paykel) CPAP mask at pressures between 5 and 16 cm H₂O. At the same time, this arrangement of tubular exhaust apertures is about 3 to 4 dBA quieter than the BREVIDA™ mask at 10 cm H₂O pressure. Without wishing to be bound by any theory, the use of tubular exhaust apertures is believed to smooth what is a noisy turbulent airflow to a quieter, more laminar, flow.

As shown in FIG. 30, the entire flow path length is accommodated by the wall thickness of the substantially rigid housing. If additional flow path length is desired or the wall thickness is reduced, these tubular exhaust apertures could be constructed to extend externally or internally from the wall in the form of actual tubular structures.

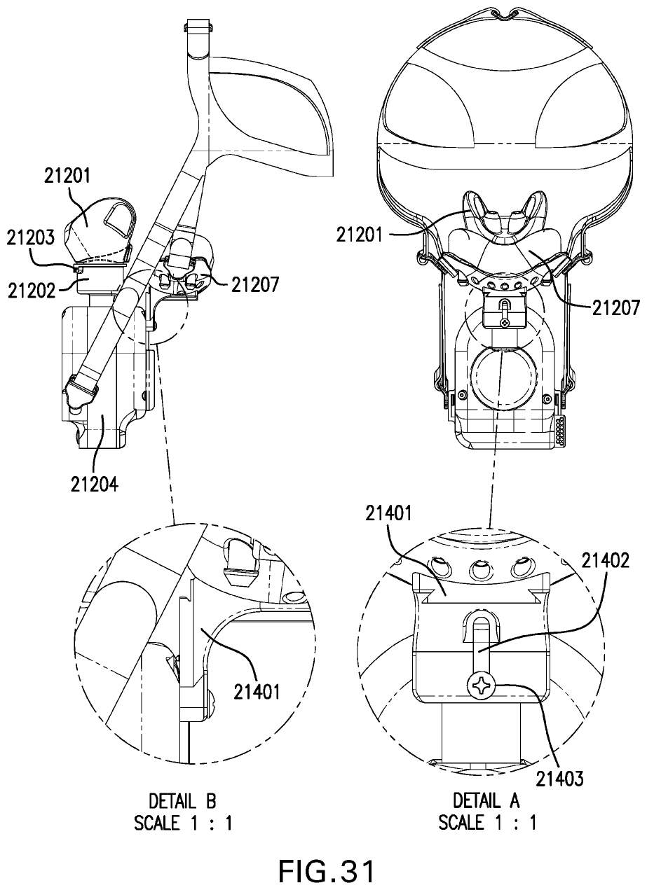

In the embodiment depicted in FIG. 31, the chin cup 21207 is advantageously mounted via a dovetail rail system that allows adjustment of the chin cup along two axes as shown. That is, the chin cup 21207 may slide along the dovetail rail 21401 in a direction that runs up and down relative to the pressurized gas source 21204, and may slide fore and aft relative to the pressurized gas source 21204 by means of slot 21402.

The dovetail rail system may then be fixed into a final position by tightening screw 21403. In this way, the chin cup can be adjusted to fit a number of different facial shapes and sizes and also may be removed and replaced.

In preliminary patient testing, users of the nasal interface systems described herein have reported an improvement in the Apnea-Hypopnea Index (AHI) and more sleep/usage hours than with their regular nasal CPAP mask They also reported feeling much better when they wake up and much easier fitting of the mask and refitting during the night. Users who did not use a heated humidifier reported comfort and effectiveness of the humidifier insert and experienced less nasal congestion in the morning. Finally, users reported a quieter CPAP experience.

Aspects of the devices described herein may be formed using a number of methods known to those of skill in the art, including but not limited to injection molding, machining, etching, 3D printing, etc. In preferred embodiments, the elements of the oronasal interface system are injection molded, a process for forming thermoplastic and thermoset materials into molded products of intricate shapes, at high production rates and with good dimensional accuracy.

The resilient elastomeric portions of the devices described herein may be formed, molded, or fabricated from any suitable material or combination of materials. Non-limiting examples of such materials include, but are not limited to, thermoplastic elastomers (TPEs), silicone, rubber, copolyesters, polycarbonates, or urethane. Examples of TPE products include block copolymers such as Thermolast, Hipex, Copec, For Tec E, Santoprene, Termoton, Arnitel, Solprene, Engage, Hytrel, Dryflex, Mediprene, Kraton, and Pibiflex; elasomeric alloys sucxh as Thermolast A, Thermolast V, Hipex, Forprene, Termoton-V and Vegaprene; styrenic block copolymers such as Thermolast K, Thermolast M, Sofprene, and Laprene; thermoplastic polyurethanes such as Copec; and thermoplastic olefins such as For-Tec E.

Portions of the devices may be made of a rigid or substantially rigid material. The skilled artisan will understand that a number of polymers may be used including thermoplastics, some thermosets, and elastomers. Thermoplastic materials become flowing liquids when heated and solids when cooled, they are often capable of undergoing multiple heating/cooling cycles without losing mechanical properties.

Thermoset materials are made of prepolymers which upon reaction cure irreversibly into a solid polymer network. Elastomers are viscoelastic materials which exhibit both elastic and viscous properties and can be either a thermoplastic or thermoset. Common thermoplastics include PMMA, cyclic olefin copolymer, ethylene vinyl acetate, polyacrylate, polyaryletherketone, polybutadiene, polycarbonate, polyester, polyetherimide, polysulfone, nylon, polyethylene, and polystyrene. Common thermosets include polyesters, polyurethanes, duroplast, epoxy resins, and polyimides. This list is not meant to be limiting. Functional filler materials such as talc and carbon fibers can be included for purposes of improving stiffness, working temperatures, and part shrinkage.

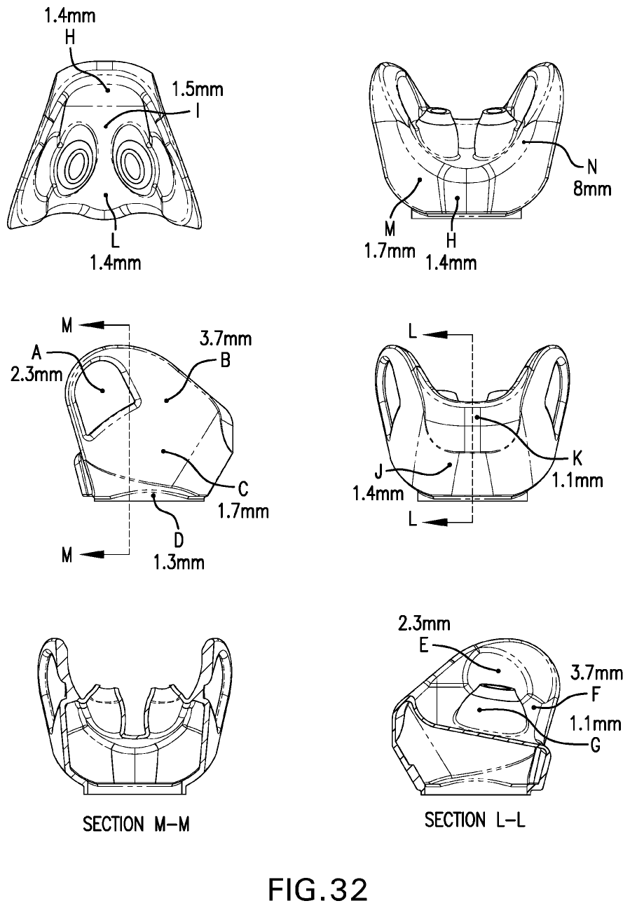

The devices of the present invention are preferably sufficiently stiff, and exhibit additional material thickness regionally, such that the devices reduce deformation in use as compared to various prior art masks that can deform/collapse/move and leak as the user moves during sleep. FIG. 32 depicts wall thicknesses (in mm) at various points on the nasal interface. If all other parameters such as shape and material durometer remain roughly the same, a silicone rubber section twice as thick should be eight times as stiff, as stiffness is proportional to the cube of the thickness based on the following equation:
Flexural Stiffness=qEt³/12s where q and s are constants for the material and shape, E is Young's Modulus, related to Durometer, and t is thickness. (From E. Watanabe, et al, Analysis and Design of Plated Structures, second edition 2022).

Exemplary regional wall thicknesses for a nasal interface are shown in FIG. 32. For the critical device areas sealing under the nose (areas labeled I and L) thicknesses of ~1.5 mm are ~4 to 5 times thicker (−50 to 100 times stiffer) than the corresponding thicknesses of ~0.3 mm on comparable areas of Fisher and Paykel Healthcare Pilairo and Brevida masks. For the wall thicknesses at the side of the nose adjacent to the outer sides of the nasal prongs, the respective measurements for area N were 7.8 mm, as compared to 3.2 mm for the Pilairo and Brevida masks. As such, these areas are 2.5 times thicker and so 15 times stiffer. The I and L stiffness regions are particularly relevant for stopping the overall deformation of the mask and collapse of the nasal prongs during use, and the N area regional stiffness in combination with the contained nasal prongs, is particularly relevant to stopping the mask moving during use and also allowing easy user fitment by instantly holding the nasal prongs firmly and correctly in the right position. These thicknesses are approximate and exemplary only.

The following are exemplary embodiments of the invention
1. An oronasal patient interface system adapted for the delivery of positive air pressure to an individual, comprising:
   a nasal interface portion comprising a first resilient elastomeric hollow chamber body defining a first space-filled interior chamber, the first hollow chamber body comprising
      a concave, bowl-shaped, skin contact surface configured to receive the nose of the individual, wherein the skin contact surface comprises a superior margin configured to contact the individual's skin at the supratip lobule and terminate contact of the unitary chamber with the individual's skin at or inferior to the supratip break, and an inferior margin configured to contact the individual's skin inferior to the coumella and superior to the upper vermillion border,
      a surface recess in the skin contact surface positioned inferior to the superior margin and configured to receive the individual's tip lobule,
      a pair of nasal interfaces, each nasal interface comprising an opening at an upper surface thereof in fluid communication with the space-filled interior chamber, and
      first and second elastomeric side members, each side member configured to extend laterally from the resilient elastomeric hollow chamber body and comprising an interior surface recess in the second surface region configured to receive the individual's alar rim, wherein each side member is configured to contact the individual's skin and extend over a nasal ala towards a position overlying the maxilla and terminate contact of the first hollow chamber body with the individual's skin proximal to the zygomaticomaxillary suture;
an oral interface portion configured to define a plenum space over the mouth of the individual, the oral interface comprising a perimeter surface providing a seal-forming structure configured to form a seal with a region of the patient's face surrounding an entrance to the patient's airways and extend around and under the mental protuberance of the individual to envelop the chin of the individual; and
a gas ingress port configured to operably connect to a source of pressurized gas,
   wherein the plenum space and the hollow chamber body are each configured in fluid communication with the gas ingress port and with each other such that pressurized gas entering the unitary apparatus through the gas ingress port is delivered to the individual's airway via the plenum space and the nasal interfaces in the hollow chamber body.
2. An oronasal patient interface system according to embodiment 1, wherein each nasal interface is formed as a hollow, approximately conical frustra extending from the skin contact surface and configured to insert into a nostril.
3. An oronasal patient interface system according to embodiment 1 or 2, wherein the nasal interface portion and the oral interface portion are a single unitary element.
4. An oronasal patient interface system according to one of embodiments 1-3, further comprising a unitary substantially rigid housing comprising a first surface configured to mate to the gas ingress port and a second surface configured to operably connect to a gas source, thereby providing a fluidly connected pathway at a location between the gas source and the gas ingress port.
5. An oronasal patient interface system according to embodiment 4, wherein the unitary substantially rigid housing comprises one or more exhaust apertures that fluidly connect the plenum space and the hollow chamber body to ambient external atmosphere.
6. An oronasal patient interface system according to embodiment 5, wherein one or more of the exhaust apertures in the unitary substantially rigid housing comprise a flow path length of from about 3 mm to about 7 mm.
7. An oronasal patient interface system according to embodiment 6, wherein one or more of the exhaust apertures in the unitary substantially rigid housing comprise a flow path length of between about 3 mm to about 7 mm and an open bore diameter of between about 0.7 mm to about 2.5 mm.
8. An oronasal patient interface system according to one of embodiments 5-7, wherein the one or more exhaust apertures provide a total aperture area of between about 15 mm 2 to about 30mm².
9. An oronasal patient interface system according to embodiment 8, wherein the number of exhaust apertures in the unitary substantially rigid housing is between 4 and 60.
10. An oronasal patient interface system according to one of embodiments 5-9, wherein the number of exhaust apertures in the unitary substantially rigid housing is 17, the open bore diameter of each of the exhaust apertures in the unitary substantially rigid housing is about 1.2 mm, and the flow path length of each of the exhaust apertures in the unitary substantially rigid housing is about 5 mm.
11. An oronasal patient interface system according to one of embodiments 5-9, wherein the flow path through the one or more of the exhaust apertures in the unitary substantially rigid housing is linear.
12. An oronasal patient interface system according to one of embodiments 6-11, wherein the unitary substantially rigid housing further comprises a plurality of mount points configured to mate to a headgear configured to retain the oronasal interface system on the head of the user thereof.
13. An oronasal patient interface system according to one of embodiments 4-12, wherein the unitary substantially rigid housing further comprises four headgear mount points.
14. An oronasal patient interface system according to embodiment 13, further comprising the headgear, wherein the headgear comprises four fasteners configured to mate to the four mount points.
15. An oronasal patient interface system according to one of embodiments 1-14, further comprising a replaceable hygroscopic condenser humidifier element positioned within the unitary substantially rigid housing and configured to increase the humidity of the gas as it traverses the fluidly connected pathway between the gas source and the gas ingress port.
16. An oronasal patient interface system according to one of embodiments 1-15, wherein the nasal interface portion and the oral interface portion are formed of a silicone elastomer.
17. An oronasal patient interface system according to one of embodiments 1-15, wherein the nasal interface portion and the oral interface portion are formed of a polyurethane elastomer.
18. An oronasal patient interface system according to one of embodiments 1-15, wherein the nasal interface portion and the oral interface portion are formed of a polyurethane siloxane copolymer elastomer.
19. An oronasal patient interface system according to one of embodiments 1-18, wherein the opening at the upper surface of each nasal interface is oval, stadium, pyriform, spherical, or elliptical in shape.
20. An oronasal interface system according to one of embodiments 1-19, wherein introduction of positive air pressure in the range of 4-30 cm H₂O into the space-filled interior chamber increases the seal of each nasal interface within the nostril.
21. An oronasal interface system according to one of embodiments 1-20, wherein the oral interface portion comprises bilateral resilient flaps on the sides thereof.
22. A method of treating sleep apnea, snoring, or another breathing disorder in an individual, comprising:
   positioning the oronasal interface system according to one of embodiments 1-21 over the nose and mouth of the individual such that the nasal interfaces insert into or onto the nostrils of an individual and the oral interface portion defines a plenum space over the mouth of the individual and extends around and under the mental protuberance of the individual to envelop said mental protuberance such that the chin of the individual seats into a cup-shaped region formed by the lower portion of the oronasal interface system defining the plenum space; and
   energizing a flow generator operably connected to the oronasal interface system to administer airflow produced by the flow generator into the individual's oral cavity and nostrils via the oronasal interface system.
23. A method of administering positive airway pressure to an individual, comprising:
   positioning the oronasal interface system according to one of embodiments 1-21 over the nose and mouth of the individual such that the nasal interfaces insert into or onto the nostrils of an individual and the oral interface portion defines a plenum space over the mouth of the individual and extends around and under the mental protuberance of the individual to envelop said mental protuberance such that the chin of the individual seats into a cup-shaped region formed by the lower portion of the oronasal interface system defining the plenum space; and
   energizing a flow generator operably connected to the oronasal interface system to administer airflow produced by the flow generator into the individual's oral cavity and nostrils via the oronasal interface system.
24. A nasal interface system for delivery of positive air pressure to an individual, comprising:
   a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
      wherein the first surface region comprises
      a superior margin configured to contact the individual's skin at the supratip lobule and terminate contact of the unitary chamber with the individual's skin at or inferior to the supratip break,
      an inferior margin configured to contact the individual's skin inferior to the coumella and terminate contact of the unitary chamber with the individual's skin at or superior to the upper vermillion border,
      a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
      a surface recess in the first surface region positioned inferior to the upper margin and configured to receive the individual's tip lobule;
   first and second hollow elastomeric side members, each side member configured to extend laterally from the resilient elastomeric hollow chamber body such that the hollow interior of the elastomeric side members is fluidly continuous with the hollow interior of the hollow chamber body, wherein each side member comprises
      a third surface region configured to contact the individual's skin and extend over a nasal ala towards a position overlying the maxilla and terminate contact of the unitary chamber with the individual's skin proximal to the zygomaticomaxillary suture, and
      an interior surface recess in the second surface region configured to receive the individual's alar rim; and
   one or more exhaust apertures through the chamber body that fluidly connect the space-filled interior chamber to ambient external atmosphere.
25. A nasal interface system for delivery of positive air pressure to an individual, comprising:
   a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
      wherein the first surface region comprises
      a superior margin configured to contact the individual's skin at the supratip lobule and terminate contact of the unitary chamber with the individual's skin at or inferior to the supratip break,
      an inferior margin configured to contact the individual's skin inferior to the coumella and terminate contact of the unitary chamber with the individual's skin at or superior to the upper vermillion border,
      a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
      a surface recess in the first surface region positioned inferior to the upper margin and configured to receive the individual's tip lobule;
   first and second solid elastomeric side members, each side member configured to extend laterally from the resilient elastomeric hollow chamber body, wherein each side member comprises
      a third surface region configured to contact the individual's skin and extend over a nasal ala towards a position overlying the maxilla and terminate contact of the unitary chamber with the individual's skin proximal to the zygomaticomaxillary suture,
      an interior surface recess in the second surface region configured to receive the individual's alar rim, and
      a fourth surface region configured to face distally from the individual's skin and to provide a region of increased flexibility in the portion of the side member overlying the maxilla; and
   one or more exhaust apertures through the chamber body that fluidly connect the space-filled interior chamber to ambient external atmosphere.
26. A nasal interface system according to embodiment 24 or 25, further comprising a unitary substantially rigid housing comprising a first surface configured to mate to the gas ingress port and a second surface configured to operably connect to a gas source, thereby providing a fluidly connected pathway between the gas source and the gas ingress port.
27. A nasal interface system according to embodiment 26, wherein the unitary substantially rigid housing further comprises shielding portions extending across a portion of an external surface of the chamber body distal to the first surface region.
28. A nasal interface system according to embodiment 27, wherein the unitary substantially rigid housing further comprises a plurality of mount points configured to mate to a headgear configured to retain the nasal interface system on the head of the user thereof.
29. A nasal interface system according to embodiment 28, wherein the unitary substantially rigid housing further comprises four mount points.
30. A nasal interface system according to embodiment 29, further comprising the headgear, wherein the headgear comprises four fasteners configured to mate to the four mount points.
31. A nasal interface system according to one of embodiments 24-30, further comprising a replaceable hygroscopic condenser humidifier element positioned within the unitary substantially rigid housing and configured to increase the humidity of the gas as it traverses the fluidly connected pathway between the gas source and the gas ingress port.
32. A nasal interface system according to one of embodiments 24-31, wherein the resilient elastomeric chamber body is configured such that introduction of positive air pressure in the range of 4-30 cm H₂O into the space-filled interior chamber causes the space-filled interior chamber to increase in volume.
33. A nasal interface system according to one of embodiments 24-32, wherein the chamber body is formed of a silicone elastomer.
34. A nasal interface system according to one of embodiments 24-32, wherein the chamber body is formed of a polyurethane elastomer.
35. A nasal interface system according to one of embodiments 24-32, wherein the chamber body is formed of a polyurethane siloxane copolymer elastomer.
36. A nasal interface system according to one of embodiments 24-35, wherein the portion of the chamber body forming the space-filled interior chamber has a Shore A durometer of between 10 and 80.
37. A nasal interface system according to embodiment 36, wherein the portion of the chamber body forming the space-filled interior chamber has a Shore A durometer of between 30 and 60 and a wall thickness of between 0.5 mm and 2.0 mm.
38. A nasal interface system according to one of embodiments 24-37, wherein the opening at the upper surface of each nasal interface is oval, stadium, pyriform, spherical, or elliptical in shape.
39. A nasal interface system according to one of embodiments 24-38, wherein the chamber body retains its shape, and a force of 4 N collapses, the portion of the chamber body forming the space-filled interior chamber when the space-filled interior chamber is at 1 atm of pressure.
40. A nasal interface system according to one of embodiments 24-39, wherein the skin contact surface of the chamber body is configured to terminate proximal to the infraorbital portion of the face of the individual between the first and second margins.
41. A nasal interface system according to one of embodiments 24-40, wherein introduction of positive air pressure in the range of 4-30 cm H₂O into the space-filled interior chamber increases the seal of each nasal interface within the nostril.
42. A method of treating sleep apnea, snoring, or another breathing disorder in an individual, comprising:
   positioning the nasal interface system according to one of embodiments 24-41 over the nose of the individual such that the nasal interfaces, each formed as a hollow, approximately conical, frustra extending from the skin contact surface, insert into or onto the nostrils of an individual; and
   energizing a flow generator operably connected to the nasal interface system to administer airflow produced by the flow generator into the individual's nostrils via the nasal interface system.
43. A respiratory mask configured for the delivery of positive pressure gas to an individual, comprising:
   an interior surface defining a plenum space configured and arranged to position over the nose, mouth, or nose and mouth of the individual and to contact the face of the individual with portion of the respiratory mask providing a peripheral seal; and
   a sensor module comprising a photoplethysmogram (PPG) sensor comprising a light emitter and a light detector configured to generate PPG sensor data, wherein the sensor module is configured and arranged to be positioned separated from the skin surface of the individual by the peripheral seal and to collect PPG sensor data through the peripheral seal.
44. A respiratory mask configured for the delivery of positive pressure gas to an individual, comprising:
   a resilient oral or oronasal patient interface; and
   a frame comprising a first aperture configured to receive the positive pressure gas from a gas source and a second aperture configured to operably connect to the resilient oral or oronasal patient interface to administer the positive pressure gas to the individual,
   wherein the oral or oronasal patient interface system comprises one or more exhaust apertures in the form of elongate hollow tubes comprising a flow path length of from about 3 mm to about 7 mm and an open bore diameter of between about 0.7 mm to about 2.5 mm.
45. A respiratory mask configured for the delivery of positive pressure gas to an individual, comprising:
   an elastomeric patient interface configured and arranged to position over the mouth of an individual and comprising interior surface defining a plenum space; and
   a pair of resilient flaps external to the plenum space and configured and arranged to lie on the cheeks (buccæ) on the sides of the face or the individual,
   wherein the pair of resilient flaps are unitary with the elastomeric patient interface.

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

Those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims:

1. An oronasal patient interface system adapted for the delivery of positive air pressure to an individual, comprising:
a nasal interface portion comprising a first resilient elastomeric hollow chamber body defining a first space-filled interior chamber, the first hollow chamber body comprising
a concave, bowl-shaped, skin contact surface configured to receive the nose of the individual, wherein the skin contact surface comprises a superior margin configured to contact the individual's skin at the supratip lobule and terminate contact of the unitary chamber with the individual's skin at or inferior to the supratip break, and an inferior margin configured to contact the individual's skin inferior to the coumella and superior to the upper vermillion border,
a surface recess in the skin contact surface positioned inferior to the superior margin and configured to receive the individual's tip lobule,
a pair of nasal interfaces, each nasal interface comprising an opening at an upper surface thereof in fluid communication with the space-filled interior chamber, and
first and second elastomeric side members, each side member configured to extend laterally from the resilient elastomeric hollow chamber body and comprising an interior surface recess in the second surface region configured to receive the individual's alar rim, wherein each side member is configured to contact the individual's skin and extend over a nasal ala towards a position overlying the maxilla and terminate contact of the first hollow chamber body with the individual's skin proximal to the zygomaticomaxillary suture;
an oral interface portion configured to define a plenum space over the mouth of the individual, the oral interface comprising a perimeter surface providing a seal-forming structure configured to form a seal with a region of the patient's face surrounding an entrance to the patient's airways and extend around and under the mental protuberance of the individual to envelop the chin of the individual; and
a gas ingress port configured to operably connect to a source of pressurized gas,
wherein the plenum space and the hollow chamber body are each configured in fluid communication with the gas ingress port and with each other such that pressurized gas entering the unitary apparatus through the gas ingress port is delivered to the individual's airway via the plenum space and the nasal interfaces in the hollow chamber body.

2. An oronasal patient interface system according to claim 1, wherein each nasal interface is formed as a hollow, approximately conical frustra extending from the skin contact surface and configured to insert into a nostril.

3. An oronasal patient interface system according to claim 1, wherein the nasal interface portion and the oral interface portion are a single unitary element.

4. An oronasal patient interface system according to claim 1, further comprising a unitary substantially rigid housing comprising a first surface configured to mate to the gas ingress port and a second surface configured to operably connect to a gas source, thereby providing a fluidly connected pathway at a location between the gas source and the gas ingress port.

5. An oronasal patient interface system according to claim 4, wherein the unitary substantially rigid housing comprises one or more exhaust apertures that fluidly connect the plenum space and the hollow chamber body to ambient external atmosphere.

6. An oronasal patient interface system according to claim 5, wherein one or more of the exhaust apertures in the unitary substantially rigid housing comprise a flow path length of from about 3 mm to about 7 mm.

7. An oronasal patient interface system according to claim 6, wherein one or more of the exhaust apertures in the unitary substantially rigid housing comprise a flow path length of between about 3 mm to about 7 mm and an open bore diameter of between about 0.7 mm to about 2.5 mm.

8. An oronasal patient interface system according to claim 7 wherein the one or more exhaust apertures provide a total aperture area of between about 15 mm² to about 30mm².

9. An oronasal patient interface system according to claim 8, wherein the number of exhaust apertures in the unitary substantially rigid housing is between 4 and 60.

10. An oronasal patient interface system according to claim 9, wherein the number of exhaust apertures in the unitary substantially rigid housing is 17, the open bore diameter of each of the exhaust apertures in the unitary substantially rigid housing is about 1.2 mm, and the flow path length of each of the exhaust apertures in the unitary substantially rigid housing is about 5 mm, optionally wherein the flow path through the one or more of the exhaust apertures in the unitary substantially rigid housing is linear.

11. (canceled)

12. An oronasal patient interface system according to claim 10, wherein the unitary substantially rigid housing further comprises a plurality of mount points configured to mate to a headgear configured to retain the oronasal interface system on the head of the user thereof, wherein the unitary substantially rigid housing optionally further comprises four headgear mount points.

13. (canceled)

14. An oronasal patient interface system according to claim 12, further comprising headgear comprising four fasteners configured to mate to the four mount points.

15. An oronasal patient interface system according to claim 1, further comprising a replaceable hygroscopic condenser humidifier element positioned within the unitary substantially rigid housing and configured to increase the humidity of the gas as it traverses the fluidly connected pathway between the gas source and the gas ingress port.

16. An oronasal patient interface system according to claim 1, wherein the nasal interface portion and the oral interface portion are formed of a silicone elastomer, a polyurethane elastomer, or a polyurethane siloxane copolymer elastomer.

17. (canceled)

18. (canceled)

19. An oronasal patient interface system according to claim 1, wherein the opening at the upper surface of each nasal interface is oval, stadium, pyriform, spherical, or elliptical in shape.

20. An oronasal interface system according to claim 1, wherein introduction of positive air pressure in the range of 4-30 cm H₂O into the space-filled interior chamber increases the seal of each nasal interface within the nostril.

21. An oronasal interface system according to claim 1, wherein the oral interface portion comprises bilateral resilient flaps on the sides thereof.

22. A method of treating sleep apnea, snoring, or another breathing disorder in an individual, comprising:
positioning the oronasal interface system according to claim 1 over the nose and mouth of the individual such that the nasal interfaces insert into or onto the nostrils of an individual and the oral interface portion defines a plenum space over the mouth of the individual and extends around and under the mental protuberance of the individual to envelop said mental protuberance such that the chin of the individual seats into a cup-shaped region formed by the lower portion of the oronasal interface system defining the plenum space; and
energizing a flow generator operably connected to the oronasal interface system to administer airflow produced by the flow generator into the individual's oral cavity and nostrils via the oronasal interface system.

23. A method of administering positive airway pressure to an individual, comprising:
positioning the oronasal interface system according to claim 1 over the nose and mouth of the individual such that the nasal interfaces insert into or onto the nostrils of an individual and the oral interface portion defines a plenum space over the mouth of the individual and extends around and under the mental protuberance of the individual to envelop said mental protuberance such that the chin of the individual seats into a cup-shaped region formed by the lower portion of the oronasal interface system defining the plenum space; and
energizing a flow generator operably connected to the oronasal interface system to administer airflow produced by the flow generator into the individual's oral cavity and nostrils via the oronasal interface system.

24. A nasal interface system for delivery of positive air pressure to an individual, comprising:
a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
wherein the first surface region comprises
a superior margin configured to contact the individual's skin at the supratip lobule and terminate contact of the unitary chamber with the individual's skin at or inferior to the supratip break,
an inferior margin configured to contact the individual's skin inferior to the coumella and terminate contact of the unitary chamber with the individual's skin at or superior to the upper vermillion border,
a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
a surface recess in the first surface region positioned inferior to the upper margin and configured to receive the individual's tip lobule;
first and second hollow elastomeric side members, each side member configured to extend laterally from the resilient elastomeric hollow chamber body such that the hollow interior of the elastomeric side members is fluidly continuous with the hollow interior of the hollow chamber body, wherein each side member comprises
a third surface region configured to contact the individual's skin and extend over a nasal ala towards a position overlying the maxilla and terminate contact of the unitary chamber with the individual's skin proximal to the zygomaticomaxillary suture, and
an interior surface recess in the second surface region configured to receive the individual's alar rim; and
one or more exhaust apertures through the chamber body that fluidly connect the space-filled interior chamber to ambient external atmosphere.

25. A nasal interface system for delivery of positive air pressure to an individual, comprising:
a resilient elastomeric hollow chamber body defining a space-filled interior chamber, the hollow chamber body comprising (i) a first surface region forming a concave, bowl-shaped, skin contact surface configured to receive the nose of the individual, and (ii) a second surface region of the chamber body comprising a gas ingress port configured to receive a gas under positive pressure for delivery to the individual, wherein the gas ingress port is in fluid communication with the space-filled interior chamber,
wherein the first surface region comprises
a superior margin configured to contact the individual's skin at the supratip lobule and terminate contact of the unitary chamber with the individual's skin at or inferior to the supratip break,
an inferior margin configured to contact the individual's skin inferior to the coumella and terminate contact of the unitary chamber with the individual's skin at or superior to the upper vermillion border,
a pair of nasal interfaces, each nasal interface formed as a hollow, approximately conical frustra extending from the first surface region and configured to insert into a nostril, wherein each nasal interface comprises an opening at an upper surface thereof in fluid communication with the space-filled interior chamber, and
a surface recess in the first surface region positioned inferior to the upper margin and configured to receive the individual's tip lobule;
first and second solid elastomeric side members, each side member configured to extend laterally from the resilient elastomeric hollow chamber body, wherein each side member comprises
a third surface region configured to contact the individual's skin and extend over a nasal ala towards a position overlying the maxilla and terminate contact of the unitary chamber with the individual's skin proximal to the zygomaticomaxillary suture,
an interior surface recess in the second surface region configured to receive the individual's alar rim, and
a fourth surface region configured to face distally from the individual's skin and to provide a region of increased flexibility in the portion of the side member overlying the maxilla; and
one or more exhaust apertures through the chamber body that fluidly connect the space-filled interior chamber to ambient external atmosphere.

26-42. (canceled)

43. A respiratory mask configured for the delivery of positive pressure gas to an individual, comprising:
an interior surface defining a plenum space configured and arranged to position over the nose, mouth, or nose and mouth of the individual and to contact the face of the individual with portion of the respiratory mask providing a peripheral seal; and
a sensor module comprising a photoplethysmogram (PPG) sensor comprising a light emitter and a light detector configured to generate PPG sensor data, wherein the sensor module is configured and arranged to be positioned separated from the skin surface of the individual by the peripheral seal and to collect PPG sensor data through the peripheral seal.

44. A respiratory mask configured for the delivery of positive pressure gas to an individual, comprising:
a resilient oral or oronasal patient interface; and
a frame comprising a first aperture configured to receive the positive pressure gas from a gas source and a second aperture configured to operably connect to the resilient oral or oronasal patient interface to administer the positive pressure gas to the individual,
wherein the oral or oronasal patient interface system comprises one or more exhaust apertures in the form of elongate hollow tubes comprising a flow path length of from about 3 mm to about 7 mm and an open bore diameter of between about 0.7 mm to about 2.5 mm.

45. A respiratory mask configured for the delivery of positive pressure gas to an individual, comprising:
an elastomeric patient interface configured and arranged to position over the mouth of an individual and comprising interior surface defining a plenum space; and
a pair of resilient flaps external to the plenum space and configured and arranged to lie on the cheeks (buccæ) on the sides of the face or the individual,
wherein the pair of resilient flaps are unitary with the elastomeric patient interface.
